(19) United States
(12) Patent Application Publication
(10) Pub. No.: US 2024/0122513 A1
(43) Pub. Date: Apr. 18, 2024

(54) AWAKENING EFFORT MOTION ESTIMATION DEVICE AND AWAKENING EFFORT MOTION ESTIMATION METHOD

(71) Applicant: Mitsubishi Electric Corporation, Tokyo (JP)

(72) Inventors: Natsuki TAHARA, Tokyo (JP); Takashi HIRANO, Tokyo (JP); Shintaro WATANABE, Tokyo (JP); Atsushi MATSUMOTO, Tokyo (JP)

(73) Assignee: Mitsubishi Electric Corporation, Tokyo (JP)

(21) Appl. No.: 18/277,411

(22) PCT Filed: Apr. 6, 2021
(86) PCT No.: PCT/JP2021/014559

§ 371 Date: Aug. 16, 2023

(51) Int. Cl. A61B 5/18; G06T 7/20; G06V 40/16

(52) U.S. Cl. CPC A61B 5/18; G06T 7/20; G06V 40/174

(57) ABSTRACT

An awakening effort motion estimation device includes processing circuitry configured to acquire a captured image obtained by imaging an occupant's face in a vehicle; detect, on a basis of the captured image having been acquired, two reference points for estimating a motion of the occupant's mouth, the two reference points including one point on a mask worn by the occupant, and the other point being a point based on a feature point of the occupant's face or a point different from the one point on the mask in the captured image; calculate a reference point distance between the detected two reference points; and estimate whether or not the occupant is performing an awakening effort motion by moving his or her mouth depending on whether or not the calculated reference point distance satisfies an awakening effort estimating condition.

### TECHNICAL FIELD

The present disclosure relates to an awakening effort motion estimation device and an awakening effort motion estimation method for estimating whether or not an occupant in a vehicle is performing an awakening effort motion.

### BACKGROUND ART

In general, when a person is in a state in which attention or judgment is decreased by drowsiness, drinking, fatigue, or the like (hereinafter, referred to as “awakening level decrease state”), the person notices a decrease in the awakening level and performs a motion of making an effort to awaken (hereinafter, referred to as “awakening effort motion”). The awakening effort motion includes a motion performed by moving a mouth, such as yawning.

Conventionally, in a vehicle, for example, there is a known technique of estimating whether or not an occupant is in an awakening level decrease state by estimating whether or not the occupant is performing an awakening effort motion on the basis of a motion of the occupant's mouth detected by using information of the occupant's face acquired by a passenger monitoring system (PMS) (for example, Patent Literature 1).

Note that it is known that the awakening level of a person does not monotonically change from a state in which the person is awake until the awakening level decreases and the person falls asleep, and the awakening level is increased by the person performing an awakening effort motion. By estimating an awakening level decrease state of a person depending on presence or absence of an awakening effort motion, the awakening level decrease state of the person can be estimated with high accuracy.

### CITATION LIST

### Patent Literature

Patent Literature 1: JP 2019-111090 A

### SUMMARY OF INVENTION

### Technical Problem

In related art, in a case where an occupant in a vehicle wears a mask, there is a problem that it is not possible to estimate whether or not the occupant is performing an awakening effort motion by moving his or her mouth.

The present disclosure has been made in order to solve the above problem, and an object of the present disclosure is to provide an awakening effort motion estimation device capable of estimating that an occupant is performing an awakening effort motion by moving his or her mouth even when the occupant wears a mask.

### Solution to Problem

An awakening effort motion estimation device according to the present disclosure includes: a captured image acquiring unit that acquires a captured image obtained by imaging an occupant's face in a vehicle; a reference point detecting unit to detect, on a basis of the captured image acquired by the captured image acquiring unit, two reference points for estimating a motion of the occupant's mouth, the two reference points including one point on a mask worn by the occupant, and the other point being a point based on a feature point of the occupant's face or a point different from the one point on the mask in the captured image; a distance calculating unit that calculates a reference point distance between the two reference points detected by the reference point detecting unit; and a motion estimating unit that estimates whether or not the occupant is performing an awakening effort motion by moving his or her mouth depending on whether or not the reference point distance calculated by the distance calculating unit satisfies an awakening effort estimating condition.

### Advantageous Effects of Invention

According to the present disclosure, it is possible to estimate that an occupant is performing an awakening effort motion by moving his or her mouth even when the occupant wears a mask.

### BRIEF DESCRIPTION OF DRAWINGS

FIG. 1 is a diagram illustrating a configuration example of an awakening effort motion estimation device according to a first embodiment.

FIGS. 2A, 2B, and 2C are each a diagram for describing a pattern of motion of a mask in a captured image in a case where a driver moves his or her mouth while wearing the mask in the first embodiment.

FIG. 3 is a flowchart for describing an operation of the awakening effort motion estimation device according to the first embodiment.

FIGS. 4A and 4B are each a diagram illustrating an example of a hardware configuration of the awakening effort motion estimation device according to the first embodiment.

FIG. 5 is a diagram illustrating a configuration example of an awakening effort motion estimation device according to a second embodiment.

FIG. 6 is a flowchart for describing an operation of the awakening effort motion estimation device according to the second embodiment.

FIG. 7 is a diagram illustrating a configuration example of an awakening effort motion estimation device according to a third embodiment.

FIG. 8 is a flowchart for describing an operation of the awakening effort motion estimation device according to the third embodiment.

FIG. 9 is a diagram illustrating a configuration example of an awakening effort motion estimation device according to a fourth embodiment.

FIG. 10 is a flowchart for describing an operation of the awakening effort motion estimation device according to the fourth embodiment.

### DESCRIPTION OF EMBODIMENTS

Hereinafter, embodiments of the present disclosure will be described in detail with reference to the drawings.

### First Embodiment

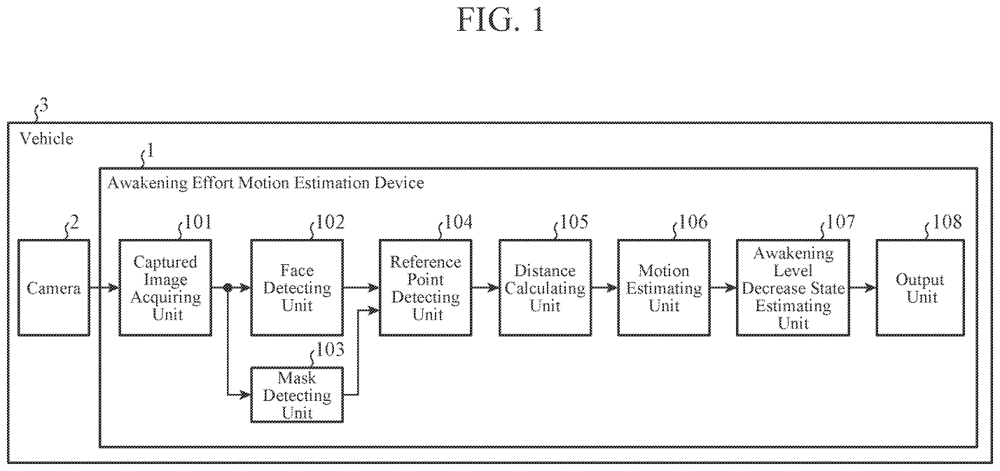

FIG. 1 is a diagram illustrating a configuration example of an awakening effort motion estimation device 1 according to a first embodiment.

The awakening effort motion estimation device 1 according to the first embodiment is assumed to be mounted on a vehicle 3.

The awakening effort motion estimation device 1 is connected to a camera 2.

The camera 2 is mounted on the vehicle 3. The camera 2 is disposed in a central portion of an instrument panel of the vehicle 3, a meter panel thereof, or the like for the purpose of monitoring a vehicle interior. The camera 2 is disposed so as to be able to image at least an occupant's face. In the first embodiment, the camera 2 is assumed to be shared with a so-called passenger monitoring system (PMS).

The camera 2 is a visible light camera or an infrared camera. In a case where the camera 2 is an infrared camera, the infrared camera includes a light source (not illustrated) that emits infrared rays for imaging to a range including an occupant's face. The light source is constituted by, for example, a light emitting diode (LED).

Note that only one camera 2 is disposed in the vehicle 3 in FIG. 1, but this is merely an example. A plurality of cameras 2 may be disposed in the vehicle 3.

The camera 2 outputs an image obtained by imaging (hereinafter, referred to as “captured image”) to the awakening effort motion estimation device 1.

The awakening effort motion estimation device 1 estimates whether or not an occupant wearing a mask is performing an awakening effort motion by moving his or her mouth on the basis of the captured image acquired from the camera 2. In the first embodiment, it is presupposed that the occupant in the vehicle 3 wears a mask.

In addition, the awakening effort motion estimation device 1 estimates an awakening level decrease state of an occupant wearing a mask on the basis of an awakening effort estimating result of whether or not the occupant is performing an awakening effort motion by moving his or her mouth.

Note that, in the following first embodiment, the occupant is assumed to be the driver of the vehicle 3. However, this is merely an example, and the awakening effort motion estimation device 1 can also estimate whether or not an occupant other than the driver of the vehicle 3 is performing an awakening effort motion by moving his or her mouth.

Hereinafter, the driver of the vehicle 3 is also simply referred to as “driver”. In addition, hereinafter, the awakening effort motion by moving a mouth is also simply referred to as “awakening effort motion”.

The awakening effort motion estimation device 1 includes a captured image acquiring unit 101, a face detecting unit 102, a mask detecting unit 103, a reference point detecting unit 104, a distance calculating unit 105, a motion estimating unit 106, an awakening level decrease state estimating unit 107, and an output unit 108.

The captured image acquiring unit 101 acquires a captured image from the camera 2.

The captured image acquiring unit 101 outputs the acquired captured image to the face detecting unit 102 and the mask detecting unit 103.

The face detecting unit 102 detects a driver's face and detects a part of the driver's face on the basis of the captured image acquired by the captured image acquiring unit 101. Specifically, on the basis of the captured image acquired by the captured image acquiring unit 101, in the captured image, the face detecting unit 102 detects the driver's face and detects a feature point of the driver's face indicating a part of the driver's face. Note that the part of the face is the outer corner of the eye, the inner corner of the eye, the nose, the jaw, the top of the head, or the like.

For example, the face detecting unit 102 detects the feature point of the driver's face using a face detector based on a known general algorithm in which Adaboost or Casecade is combined with a Haar-Like detector. The face detector has learned a large amount of face image data in advance. In addition, for example, the face detecting unit 102 may detect the feature point of the driver's face using a general method such as so-called model fitting or Elastic Bunch Graph Matching. The face detecting unit 102 can detect the feature point of the driver's face using various known face recognition techniques on the basis of the captured image.

In the first embodiment, the feature point of the driver's face is represented by coordinates on the captured image.

In the captured image acquired by the captured image acquiring unit 101, the face detecting unit 102 imparts, to the feature point of the driver's face, information capable of specifying which part of the face the feature point indicates, and outputs, to the reference point detecting unit 104, a captured image after imparting to the feature point of the driver's face, the information capable of specifying which part of the face the feature point indicates (hereinafter, referred to as “captured image with a facial feature point”).

Note that, here, the awakening effort motion estimation device 1 includes the face detecting unit 102, but this is merely an example, and the awakening effort motion estimation device 1 does not necessarily include the face detecting unit 102.

The face detecting unit 102 may be disposed at a place that can be referred to by the awakening effort motion estimation device 1 outside the awakening effort motion estimation device 1.

For example, the camera 2 may include the face detecting unit 102. In this case, the camera 2 outputs the captured image with a facial feature point to the awakening effort motion estimation device 1. The captured image acquiring unit 101 acquires the captured image with a facial feature point output from the camera 2, and outputs the acquired captured image with a facial feature point to the reference point detecting unit 104. Details of the reference point detecting unit 104 will be described later.

The mask detecting unit 103 detects a mask worn by the driver on the basis of the captured image acquired by the captured image acquiring unit 101. Specifically, the mask detecting unit 103 detects a region where the mask worn by the driver is imaged in the captured image.

The mask detecting unit 103 only needs to detect the mask worn by the driver using, for example, a known image recognition technique. For example, the mask detecting unit 103 may detect the mask worn by the driver using a face detector based on a general algorithm used by the face detecting unit 102 to detect the feature point of the driver's face. In this case, it is assumed that a large amount of face image data when the face detector performs learning includes face image data when a mask is worn. Note that examples of the mask include masks made of different types of materials, such as a nonwoven mask, a cloth mask, and a urethane mask. In addition, examples of the mask include masks of various colors. In addition, examples of the mask include a patterned mask and a non-patterned mask. When a face detector is used to detect the mask worn by the driver, it is preferable to cause the face detector to learn face image data wearing various types of masks in advance.

In the first embodiment, the mask worn by the driver is represented by a region on the captured image.

In the captured image acquired by the captured image acquiring unit 101, the mask detecting unit 103 imparts, to the region of the mask worn by the driver, information capable of specifying the region of the mask, and outputs, to the reference point detecting unit 104, a captured image after imparting to the region of the mask worn by the driver, the information capable of specifying the region of the mask (hereinafter, referred to as “captured image with a mask region”).

Note that, here, the awakening effort motion estimation device 1 includes the mask detecting unit 103, but this is merely an example, and the awakening effort motion estimation device 1 does not necessarily include the mask detecting unit 103.

The mask detecting unit 103 may be disposed at a place that can be referred to by the awakening effort motion estimation device 1 outside the awakening effort motion estimation device 1.

For example, the camera 2 may include the mask detecting unit 103. In this case, the camera 2 outputs the captured image with a mask region to the awakening effort motion estimation device 1. The captured image acquiring unit 101 acquires the captured image with a mask region output from the camera 2, and outputs the acquired captured image with a mask region to the reference point detecting unit 104. Details of the reference point detecting unit 104 will be described later.

On the basis of the captured image acquired by the captured image acquiring unit 101, in the captured image, the reference point detecting unit 104 detects two reference points for estimating a motion of the driver's mouth, the two reference points being a point on the mask worn by the driver, the other point being a point based on a feature point of the driver's face or a point different from the one point on the mask.

More specifically, on the basis of the captured image with a facial feature point output from the face detecting unit 102 and the captured image with a mask region output from the mask detecting unit 103, in the captured image, the reference point detecting unit 104 detects two reference points for estimating a motion of the driver's mouth, the two reference points being a point on a mask worn by the driver, the other point being a point based on a feature point of the driver's face or a point different from the one point on the mask.

When the driver wearing the mask moves his or her mouth, the mask moves with the motion of the mouth. The awakening effort motion estimation device 1 can estimate the motion of the mouth moved by the driver wearing the mask by focusing on how the mask worn by the driver moves in the captured image. The reference point detecting unit 104 detects the two reference points in the captured image so as to be able to detect the motion of the mask in a case the driver wearing the mask moves his or her mouth.

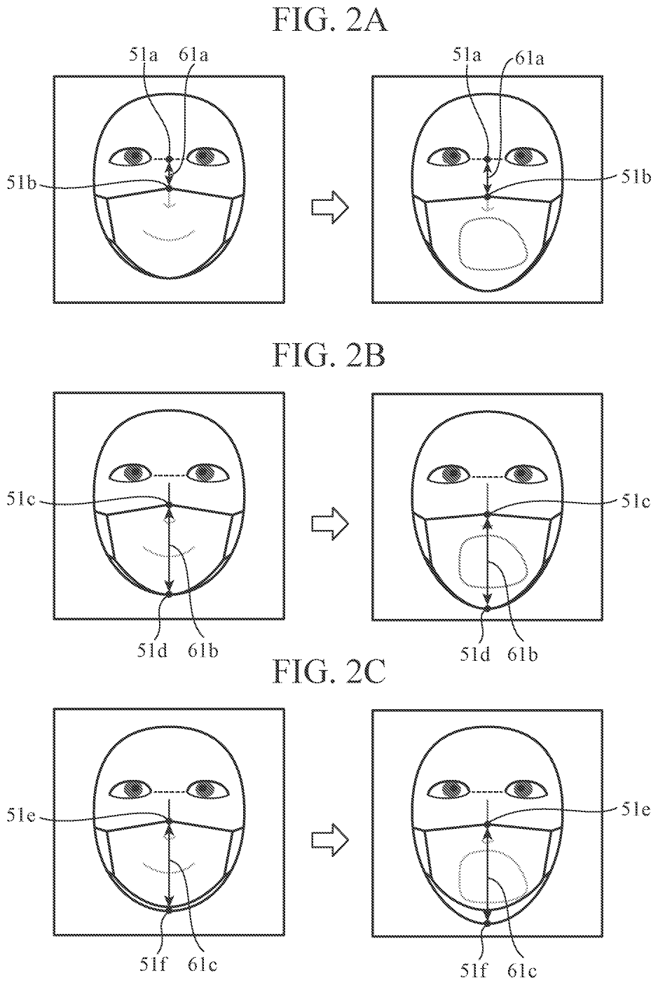

Here, FIGS. 2A, 2B, and 2C are each a diagram for describing a pattern of motion of a mask in the captured image in a case where the driver moves his or her mouth while wearing the mask.

Note that the faces illustrated in FIGS. 2A, 2B, and 2C are driver's faces. In a case where the driver moves his or her mouth while wearing the mask, a pattern as illustrated in FIG. 2A, 2B, or 2C is assumed as a pattern of the motion of the mask. Note that, in FIGS. 2A, 2B, and 2C, as an example of the motion of the driver's mouth, a motion in which the driver opens his or her mouth up and down is illustrated. For example, when the driver yawns, the driver's mouth opens up and down.

FIG. 2A is a diagram illustrating a pattern of motion of the mask in which the mask is pulled by the jaw and an upper end of the mask is lowered when the driver moves his or her mouth. This pattern is hereinafter referred to as “pattern A”.

FIG. 2B is a diagram illustrating a pattern of motion of the mask in which the mask itself extends up and down when the driver moves his or her mouth. In this pattern, it is assumed that the mask worn by the driver is a highly stretchable mask. This pattern is hereinafter referred to as “pattern B”.

FIG. 2C is a diagram illustrating a pattern of motion of the mask in which the driver wears the mask in a state where the mask is not caught on the jaw, and the mask is not pulled by the driver's jaw and cannot cover the driver's jaw, in other words, the driver's jaw protrudes from the mask when the driver moves his or her mouth. This pattern is hereinafter referred to as “pattern C”.

In the case of pattern A, since the upper end of the mask is lowered when the driver moves his or her mouth, a distance between one point on the driver's face not covered with the mask and one point of the upper end of the mask increases with the motion of the driver's mouth.

That is, the awakening effort motion estimation device 1 can estimate the motion of the mouth moved by the driver wearing the mask on the basis of the magnitude of the distance between one point on the driver's face not covered with the mask and one point of the upper end of the mask.

Thus, in order to estimate a motion of the driver's mouth in the case of pattern A, the awakening effort motion estimation device 1 defines, as reference points in pattern A, one point on the driver's face and one point of the upper end of the mask in the captured image, and estimates the motion of the mouth moved by the driver wearing the mask on the basis of a distance between the reference points (hereinafter, referred to as “reference point distance”).

In the first embodiment, in pattern A, the reference point that is one point on the driver's face is defined as a reference point based on a feature point of the driver's face, specifically, a feature point indicating both inner corners of the driver's eyes. More specifically, in the first embodiment, in pattern A, the reference point that is one point on the driver's face is defined as the center of both inner corners of the driver's eyes.

In addition, in the first embodiment, the one point of the upper end of the mask in pattern A is defined as an uppermost point of the upper end of the mask.

In FIG. 2A, the reference point that is one point on the driver's face in pattern A, that is, the center of both inner corners of the driver's eyes is indicated by a reference numeral 51a. In addition, in FIG. 2A, the reference point that is one point of the upper end of the mask in pattern A, that is, the uppermost point of the upper end of the mask is indicated by a reference numeral 51b. In FIG. 2A, a line segment (indicated by a reference numeral 61a in FIG. 2A) connecting the reference numerals 51a and 51b is the reference point distance.

In the case of pattern B, since the mask extends up and down when the driver moves his or her mouth, a distance between one point of the upper end of the mask and one point of a lower end of the mask increases with the motion of the driver's mouth.

That is, the awakening effort motion estimation device 1 can estimate the motion of the mouth moved by the driver wearing the mask on the basis of the magnitude of the distance between one point of the upper end of the mask and one point of the lower end of the mask.

Therefore, in order to estimate a motion of the driver's mouth in the case of pattern B, the awakening effort motion estimation device 1 defines, as reference points in pattern B, one point of the upper end of the mask worn by the driver and one point of the lower end of the mask in the captured image, and estimates the motion of the mouth moved by the driver wearing the mask on the basis of the reference point distance.

In the first embodiment, in pattern B, the reference point that is one point of the upper end of the mask worn by the driver is defined as an uppermost point of the upper end of the mask. In addition, in the first embodiment, in pattern B, the reference point that is one point of the lower end of the mask worn by the driver is defined as a lowermost point of the lower end of the mask.

In FIG. 2B, the reference point that is one point of the upper end of the mask in pattern B, that is, the uppermost point of the upper end of the mask is indicated by a reference numeral 51c. In addition in FIG. 2B, the reference point that is one point of the lower end of the mask in pattern B, that is, the lowermost point of the lower end of the mask is indicated by a reference numeral 51d. In FIG. 2B, a line segment (indicated by a reference numeral 61b in FIG. 2B) connecting the reference numerals 51c and 51d is the reference point distance.

In the case of pattern C, since the driver's jaw protrudes from the mask when the driver moves his or her mouth, a distance between one point of the upper end of the mask and a point indicating the driver's jaw increases with the motion of the driver's mouth.

That is, the awakening effort motion estimation device 1 can estimate the motion of the mouth moved by the driver wearing the mask on the basis of the magnitude of the distance between one point of the upper end of the mask and a point indicating the driver's jaw.

Therefore, in order to estimate a motion of the driver's mouth in the case of pattern C, the awakening effort motion estimation device 1 defines, as reference points in pattern C, one point of the upper end of the mask worn by the driver and a feature point indicating the driver's jaw in the captured image, and estimates the motion of the mouth moved by the driver wearing the mask on the basis of the reference point distance.

In the first embodiment, in pattern C, the reference point that is one point of the upper end of the mask worn by the driver is defined as an uppermost point of the upper end of the mask.

In FIG. 2C, the reference point that is one point of the upper end of the mask in pattern C, that is, the uppermost point of the upper end of the mask is indicated by a reference numeral 51e. In addition, in FIG. 2C, the reference point that is a feature point indicating the driver's jaw in pattern C is indicated by a reference numeral 51f. In FIG. 2C, a line segment (indicated by a reference numeral 61c in FIG. 2C) connecting the reference numerals 51e and 51f is the reference point distance.

Note that, as for the reference point in pattern C, the center of both inner corners of the driver's eyes may be used as the reference point instead of the one point of the upper end of the mask worn by the driver.

In addition, as for the reference point in pattern C, in a case where the feature point indicating the driver's jaw is not detected, one point of the lower end of the mask worn by the driver may be used as the reference point instead of the feature point indicating the driver's jaw. In this case, the one point of the lower end of the mask worn by the driver is a lowermost point of the lower end of the mask. For example, even when the driver wears the mask in a state where the mask is not caught on the jaw, it may be possible that the jaw does not protrude from the mask in a case where the driver's mouth is closed.

Note that in patterns A, B, and C, which point on the driver's face is defined as the reference point and which point on the mask worn by the driver is defined as the reference point can be set according to needs.

For example, in the first embodiment, the reference point on the driver's face is the center of both inner corners of the eyes, but this is merely an example. The reference point on the driver's face only needs to be a point based on a feature point of the driver's face. Note that the reference point on the driver's face is a stationary point on the driver's face. For example, in the captured image, the inner end of the eyebrow whose position changes by expression or the iris whose position changes by movement of a line of sight is not suitable as the reference point.

The description returns to the description using FIG. 1.

The reference point detecting unit 104 detects two reference points for each of all the plurality of patterns (patterns A, B, and C) described with reference to FIGS. 2A, 2B, and 2C.

For example, on the basis of the captured image with a facial feature point and the captured image with a mask region to which the same imaging date and time are imparted, in the captured image acquired by the captured image acquiring unit 101, the reference point detecting unit 104 generates an image in which information capable of specifying which part of the face a feature point of the driver's face indicates is imparted to the feature point, and information capable of specifying a region of the mask is imparted to the region of the mask (hereinafter, referred to as “captured image with a face mask”). The reference point detecting unit 104 detects a reference point on the basis of the captured image with a face mask. Information of the imaging date and time is imparted to the captured image.

Note that, in a case where the camera 2 includes the face detecting unit 102 and the mask detecting unit 103, the captured image with a face mask may be output from the camera 2 to the reference point detecting unit 104.

Specifically, the reference point detecting unit 104 detects the center of both inner corners of the driver's eyes and the uppermost point of the upper end of the mask as the two reference points in pattern A on the basis of the captured image with a face mask.

In addition, the reference point detecting unit 104 detects the uppermost point of the upper end of the mask and the lowermost point of the lower end of the mask as the two reference points in pattern B on the basis of the captured image with a face mask.

In addition, the reference point detecting unit 104 detects the uppermost point of the upper end of the mask and the feature point of the face indicating the driver's jaw as the two reference points in pattern C on the basis of the captured image with a face mask. Note that the reference point detecting unit 104 detects the center of both inner corners of the driver's eyes in a case where the center of both inner corners of the driver's eyes is used as the reference point instead of the uppermost point of the mask, and detects the lowermost point of the lower end of the mask in a case where the lowermost point of the lower end of the mask is used as the reference point instead of the feature point indicating the driver's jaw.

In a method for detecting the uppermost point of the upper end of the mask, for example, the reference point detecting unit 104 detects an edge of a predetermined range below an area of the driver's eyes in the captured image with a face mask, and detects an uppermost point having an edge intensity higher than a preset threshold as the uppermost point of the upper end of the mask. The size of the area of the driver's eyes in the captured image with a face mask is set depending on, for example, a width between a feature point indicating the inner corner of the eye and a feature point indicating the outer corner of the eye.

In addition, in a method for detecting the lowermost point of the lower end of the mask, for example, the reference point detecting unit 104 detects the edge of the lower area of the driver's face in the captured image with a face mask, and detects a lowermost point having an edge intensity higher than a preset threshold as the lowermost point of the lower end of the mask. The size of the lower area of the driver's face in the captured image with a face mask is set depending on, for example, the size of the driver's face. The reference point detecting unit 104 can estimate the size of the driver's face from a feature point of the driver's face.

The reference point detecting unit 104 only needs to perform edge detection using a known general edge detection filter by a Sobel method, a Gauss Laplacian method, a Canny method, or the like.

In addition, for example, when the mask detecting unit 103 detects a mask worn by the driver using a face detector based on a general algorithm, the mask detecting unit 103 may also detect a reference point in the mask. In this case, when the face detector performs learning, the face detector is caused to learn image data when the mask is worn in a state where one uppermost point of the upper end of the mask and one lowermost point of the lower end of the mask are annotated. Information capable of specifying a reference point in the mask is imparted to the captured image with a mask region output from the mask detecting unit 103, and the reference point detecting unit 104 only needs to detect the uppermost point of the upper end of the mask or the lowermost point of the lower end of the mask on the basis of the information capable of specifying the reference point in the mask.

In addition, in a method for detecting a reference point based on a feature point of the driver's face, the reference point detecting unit 104 can detect a reference point on the driver's face, in other words, the center of both inner corners of the driver's eyes or a feature point indicating the driver's jaw on the basis of a feature point of the driver's face imparted to the captured image with a face mask. Note that, for example, in a case where the face detecting unit 102 has not detected the feature point indicating the driver's jaw, the reference point detecting unit 104 may detect the point indicating the driver's jaw using a known image recognition technique, or may detect the point by edge detection of the lower area of the face.

The reference point detecting unit 104 detects the reference point for each frame of the captured images to which the same time is imparted, in other words, for each generated captured image with a face mask, output from the face detecting unit 102 and the mask detecting unit 103.

In the captured image with a face mask, the reference point detecting unit 104 imparts information capable of specifying a reference point to the reference point, and outputs a captured image with a face mask after the information capable of specifying the reference point is imparted (hereinafter, referred to as “captured image after reference point impartment”) to the distance calculating unit 105.

Note that specifically, the information capable of specifying a reference point is information capable of specifying which feature point of the driver's face the reference point is based on, or information capable of specifying which one point in the mask the reference point indicates.

Since the reference point detecting unit 104 detects a reference point for each of all the patterns A, B, and C, information capable of specifying the reference points in all the patterns A, B, and C is imparted to the captured image after reference point impartment.

Note that, here, the awakening effort motion estimation device 1 assumes all the patterns A, B, and C described above, and the reference point detecting unit 104 detects the reference points in all the patterns A, B, and C, but this is merely an example. In the first embodiment, the awakening effort motion estimation device 1 may assume only one or two of the patterns A, B, and C. In this case, the reference point detecting unit 104 only needs to detect only a reference point in a pattern of motion of the mask in a case where the driver moves his or her mouth while wearing the mask, which is assumed in the awakening effort motion estimation device 1.

The distance calculating unit 105 calculates a reference point distance between the two reference points in the captured image, detected by the reference point detecting unit 104.

More specifically, the distance calculating unit 105 calculates, on the basis of the captured image after reference point impartment output from the reference point detecting unit 104, a reference point distance between two reference points determined in each pattern in the captured image after reference point impartment for each of the patterns (here, patterns A, B, and C) of motion of the mask in a case where the driver moves his or her mouth while wearing the mask, assumed in the awakening effort motion estimation device 1. In the following first embodiment, the term “pattern” means a pattern of motion of a mask when the driver moves his or her mouth while wearing the mask.

For example, the distance calculating unit 105 calculates a Euclidean distance between two reference points, and uses the calculated Euclidean distance as a reference point distance.

In the example described with reference to FIGS. 2A, 2B, and 2C, the distance calculating unit 105 calculates a distance of a line segment indicated by the reference numeral 61a in FIG. 2A as the reference point distance of pattern A.

In addition, the distance calculating unit 105 calculates a distance of a line segment indicated by the reference numeral 61b in FIG. 2B as the reference point distance of pattern B.

In addition, the distance calculating unit 105 calculates a distance of a line segment indicated by the reference numeral 61c in FIG. 2C as the reference point distance of pattern C.

The distance calculating unit 105 outputs information regarding the calculated reference point distance (hereinafter, referred to as “reference point distance information”) to the motion estimating unit 106. The reference point distance information is, for example, a captured image after reference point impartment with which a reference point distance for each pattern is associated.

Note that, in the reference point distance information, the distance calculating unit 105 associates the calculated reference point distance with information capable of specifying a reference point distance according to which pattern is used.

On the basis of the reference point distance information output from the distance calculating unit 105, the motion estimating unit 106 estimates whether or not the driver is performing an awakening effort motion depending on whether or not the reference point distance calculated by the distance calculating unit 105 satisfies a preset condition for estimating whether or not the driver is performing the awakening effort motion (hereinafter, referred to as “awakening effort estimating condition”).

For example, the motion estimating unit 106 obtains a change amount of the reference point distance or a change period of the reference point distance in a preset time (hereinafter, referred to as “motion estimating time”), and determines whether or not the driver is performing an awakening effort motion by moving his or her mouth depending on whether or not the change amount of the reference point distance or the change period of the reference point distance satisfies the awakening effort estimating condition.

Note that, in the first embodiment, the change amount of the reference point distance simply refers to a temporal change of the reference point distance. That is, an increase in the reference point distance means that the driver is opening his or her mouth.

The motion estimating time is, for example, a time of 85 seconds or more according to needs. For example, the distance calculating unit 105 stores the reference point distance information in a storage unit (not illustrated) disposed at a place that can be referred to by the awakening effort motion estimation device, and the motion estimating unit 106 acquires the reference point distance information from before the motion estimating time to the present by referring to the storage unit, and calculates the change amount of the reference point distance and the change period of the reference point distance on the basis of the acquired reference point distance information. Then, the motion estimating unit 106 estimates whether or not the driver is performing the awakening effort motion.

As the awakening effort estimating condition, for example, a condition regarding a time-series change amount of the reference point distance or a change period of the reference point distance such as <condition 1> to <condition 5> below is set in advance.

<Condition 1>

A state in which the reference point distance is equal to or more than a preset threshold (hereinafter, referred to as “distance determination threshold”) continues for equal to or more than a preset time (hereinafter, referred to as “first determination time”).

<Condition 2>

The reference point distance changes periodically, and the periodic change of the reference point distance continues for equal to or more than a preset time (hereinafter, referred to as “second determination time”) and less than a preset time (hereinafter, referred to as “third determination time”) longer than the second determination time.

<Condition 3>

The reference point distance changes periodically, and the periodic change of the reference point distance continues for equal to or longer than the third determination time.

<Condition 4>

The reference point distance changes aperiodically.

<Condition 5>

None of <condition 1> to <condition 4> above is satisfied.

The awakening effort estimating condition is associated with information on what type of motion the driver is estimated to be performing when the condition is satisfied (hereinafter, referred to as “estimated motion type information”).

In the first embodiment, it is assumed that estimated motion type information estimating that the driver is performing an awakening effort motion by yawning is associated with <condition 1>.

In the first embodiment, when the reference point distance is equal to or more than the distance determination threshold, it is considered that the driver is opening his or her mouth wide up and down, and the motion of the driver's mouth may be a motion of yawning. That is, it can be said that the distance determination threshold is a threshold for determining that the motion of the driver's mouth is yawning (hereinafter, referred to as “yawning determination threshold”).

In addition, it is assumed that estimated motion type information estimating that the driver is performing an awakening effort motion by moving his or her mouth in a mumbling manner is associated with <condition 2>.

In addition, it is assumed that estimated motion type information estimating that the driver is eating is associated with <condition 3>.

In addition, it is assumed that estimated motion type information estimating that the driver is talking is associated with <condition 4>.

In addition, it is assumed that estimated motion type information estimating that the driver is not moving his or her mouth is associated with <condition 5>.

The first determination time in <condition 1> is, for example, three seconds. In addition, as the distance determination threshold in <condition 1>, the size of a reference point distance assumed in a case where a person with a standard face size yawns while wearing a mask is generally set in advance. The motion estimating unit 106 may set the distance determination threshold using a predetermined calculation formula on the basis of the size of the driver's face. The calculation formula is, for example, “length from the top of the head to the jaw in captured image after reference point impartment×0.2”.

The second determination time is, for example, five seconds, and the third determination time is, for example, ten seconds in <condition 2>.

Regarding the periodic change of the reference point distance set in <condition 2> and <condition 3> and the aperiodic change of the reference point distance set in <condition 4>, for example, the motion estimating unit 106 measures a peak interval of the reference point distance in the motion estimating time, and determines that the reference point distance changes periodically when a difference between the peak intervals is within plus or minus one second, and determines that the reference point distance changes aperiodically when a difference between the peak intervals is larger than plus or minus one second. Note that this is merely an example, and a method for determining that the reference point distance changes periodically or that the reference point distance changes aperiodically may be selected according to needs.

In a case where <condition 1> is satisfied, the motion estimating unit 106 estimates that the driver is performing an awakening effort motion by yawning.

In addition, in a case where <condition 2> is satisfied, the motion estimating unit 106 estimates that the driver is performing an awakening effort motion by moving his or her mouth in a mumbling manner.

In addition, in a case where <condition 3> is satisfied, the motion estimating unit 106 estimates that the driver is eating.

In addition, in a case where <condition 4> is satisfied, the motion estimating unit 106 estimates that the driver is talking.

In addition, in a case where <condition 5> is satisfied, the motion estimating unit 106 estimates that the driver is not moving his or her mouth.

As the motion in which the driver periodically moves his or her mouth, in addition to the awakening effort motion, eating in a case where the driver is awake is also conceivable.

The awakening effort motion by periodically moving the mouth, such as moving the mouth in a mumbling manner, is a motion of repeating moving the mouth and stopping the mouth several times for about five to ten seconds. Meanwhile, eating such as chewing a gum is generally a motion in which chewing is continuously performed for ten seconds or more. Therefore, as in <condition 2> and <condition 3>, whether the motion of the driver's mouth is caused by the awakening effort motion or eating is preferably estimated using an occurrence frequency of the change in the reference point distance (being a periodic change) and the change amount of the reference point distance.

As described above, the motion estimating unit 106 estimates whether or not the driver is performing the awakening effort motion, for example, from the time-series change amount of the reference point distance and the change period of the reference point distance. The motion estimating unit 106 determines that the driver is opening his or her mouth when the reference point distance increases. In addition, in a case where the reference point distance changes in the order of increase, decrease, and increase, the motion estimating unit 106 determines that the driver sequentially performs a motion of opening his or her mouth, a motion of closing his or her mouth, and then, a motion of opening his or her mouth. The motion estimating unit 106 estimates whether or not the driver is performing the awakening effort motion depending on whether such a motion of the mouth is periodic or aperiodic.

Note that the motion estimating unit 106 obtains the time-series change amount of the reference point distance and the change period of the reference point distance for the reference point distance of each pattern, and compares the change amount and the change period with the awakening effort estimating condition. Here, in a case where there is a plurality of patterns in which the reference point distance changes among patterns A, B, and C, the motion estimating unit 106 determines a reference point distance used for estimating whether or not the driver is performing the awakening effort motion on the basis of a preset priority. The priority is set by, for example, an administrator or the like depending on the type of mask that the driver is assumed to wear frequently and the manner of wearing the mask that the driver is assumed to use frequently. As a specific example, a frequency at which the driver wears a type of mask with which it is assumed that the motion of pattern A is performed when the mouth is moved, such as a nonwoven fabric, is higher than a frequency at which the driver wears a stretchable type of mask with which it is assumed that the motion of pattern B is performed. In addition, it is assumed that a frequency at which the driver wears a mask with his or her jaw protruding from the mask is extremely low. In this case, a priority is set in advance in such a manner that pattern A has the highest priority, pattern B has the second highest priority, and pattern C has the third highest priority.

In the first embodiment, as an example, it is assumed that a priority is set in such a manner that the priorities of patterns A, B, and C descend in this order.

In this case, for example, in a case where the reference point distance of pattern A and the reference point distance of pattern B both change, the motion estimating unit 106 uses the reference point distance of pattern A for estimating whether or not the driver is performing the awakening effort motion.

For example, in a case where the reference point distance of pattern B does not change for a certain period of time (for example, one hour), the motion estimating unit 106 may stop calculation of the time-series change amount of the reference point distance and the change period of the reference point distance based on the reference point distance of pattern B. For example, the manner of wearing the mask by the driver may change during driving, but stretchability of the mask does not change. Therefore, when the reference point distance of pattern B does not change for a certain period of time, it is assumed that the reference point distance does not change in the future.

In the first embodiment described above, depending on whether or not the awakening effort estimating condition is satisfied, the motion estimating unit 106 can estimate that the driver is performing the awakening effort motion, and can also estimate that the driver is performing a motion of moving his or her mouth other than the awakening effort motion (eating, talking, or not moving his or her mouth). However, this is merely an example. The motion estimating unit 106 only needs to estimate at least that the driver is performing the awakening effort motion. That is, in the awakening effort estimating condition, at least a condition that can determine whether or not the driver is performing the awakening effort motion (<condition 1> and <condition 2> in the above example) only needs to be set.

In addition, the contents of the awakening effort estimating conditions such as <condition 1> to <condition 5> described above are merely examples.

In the awakening effort estimating condition, it is only required that a condition that can be estimate that the driver is performing the awakening effort motion is set, and that the motion estimating unit 106 can estimate that the driver is performing the awakening effort motion by comparing the reference point distance with the awakening effort estimating condition.

The motion estimating unit 106 outputs a result of whether or not it has been estimated that the driver is performing the awakening effort motion (hereinafter, referred to as “awakening effort estimating result”) to the awakening level decrease state estimating unit 107.

The awakening level decrease state estimating unit 107 estimates the awakening level decrease state of the driver in consideration of the awakening effort estimating result on the basis of the awakening effort estimating result output from the motion estimating unit 106.

The awakening level decrease state estimating unit 107 estimates the awakening level decrease state of the driver using, for example, a learned model (hereinafter, referred to as a “machine learning model”).

For example, the machine learning model receives, as inputs, the captured image acquired by the captured image acquiring unit 101 and information based on the awakening effort estimating result, and outputs information indicating the awakening level decrease state. The machine learning model has performed learning in advance by so-called supervised learning.

The information based on the awakening effort estimating result as the input of the machine learning model may be, for example, a flag set to “1” in a case where it is estimated that the driver is performing the awakening effort motion and set to “0” in a case where it is estimated that the driver is not performing the awakening effort motion (hereinafter, referred to as “awakening effort motion flag”), or may be information in which the awakening effort motion flag is associated with information indicating the content of the awakening effort motion that is estimated to be performed by the driver, such as yawning.

For example, the awakening level decrease state estimating unit 107 may estimate the awakening level decrease state of the driver on the basis of a preset rule for estimating the awakening level decrease state of the driver (hereinafter, referred to as “awakening level decrease estimating rule”). The awakening level decrease estimating rule is, for example, a rule in which an awakening level decrease level is not decreased or the awakening level decrease level is decreased by one level in a case where the awakening effort motion flag is “1”.

The awakening level decrease state estimating unit 107 outputs information regarding the estimated awakening level decrease state of the driver (hereinafter referred to as “awakening level decrease state information”) to the output unit 108.

The output unit 108 outputs the awakening level decrease state information output from the awakening level decrease state estimating unit 107 to a device outside the awakening effort motion estimation device 1, such as an occupant monitoring device (not illustrated) that monitors a state of an occupant in the vehicle 3.

Note that, in the first embodiment, the awakening level decrease state estimating unit 107 and the output unit 108 are included in the awakening effort motion estimation device 1, but this is merely an example, and the awakening level decrease state estimating unit 107 and the output unit 108 are not necessarily included in the awakening effort motion estimation device 1. The awakening level decrease state estimating unit 107 and the output unit 108 may be arranged at places that can be referred to by the awakening effort motion estimation device 1 outside the awakening effort motion estimation device 1.

An operation of the awakening effort motion estimation device 1 according to the first embodiment will be described.

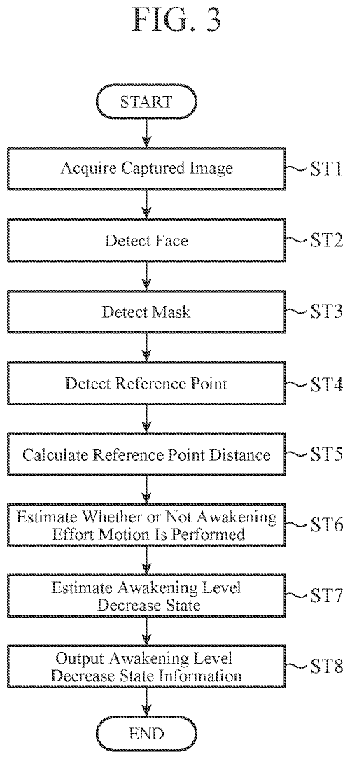

FIG. 3 is a flowchart for describing the operation of the awakening effort motion estimation device 1 according to the first embodiment.

The awakening effort motion estimation device 1 repeats the operation illustrated in the flowchart of FIG. 3, for example, from when the engine of the vehicle 3 is turned on until the engine is turned off.

The captured image acquiring unit 101 acquires a captured image from the camera 2 (step ST1).

The captured image acquiring unit 101 outputs the acquired captured image to the face detecting unit 102 and the mask detecting unit 103.

The face detecting unit 102 detects a driver's face and detects a part of the driver's face on the basis of the captured image acquired by the captured image acquiring unit 101 in step ST1 (step ST2).

The face detecting unit 102 outputs the captured image with a facial feature point to the reference point detecting unit 104.

The mask detecting unit 103 detects a mask worn by the driver on the basis of the captured image acquired by captured image acquiring unit 101 in step ST1 (step ST3).

The mask detecting unit 103 outputs the captured image with a mask region to the reference point detecting unit 104.

On the basis of the captured image acquired by the captured image acquiring unit 101 in step ST1, in the captured image, the reference point detecting unit 104 detects two reference points for estimating a motion of the driver's mouth, the two reference points including one point on a mask worn by the driver, and the other point being a point based on a feature point of the driver's face or a point different from the one point on the mask (step ST4).

More specifically, the reference point detecting unit 104 detects the two reference points on the basis of the captured image with a facial feature point output from the face detecting unit 102 in step ST2 and the captured image with a mask region output from the mask detecting unit 103 in step ST3.

The reference point detecting unit 104 outputs the captured image after reference point impartment to the distance calculating unit 105.

The distance calculating unit 105 calculates a reference point distance between the two reference points in the captured image, detected by the reference point detecting unit 104 in step ST4 (step ST5).

More specifically, the distance calculating unit 105 calculates, on the basis of the captured image after reference point impartment output from the reference point detecting unit 104, a reference point distance between two reference points determined in each pattern in the captured image after reference point impartment for each of the patterns (here, patterns A, B, and C) of motion of the mask in a case where the driver moves his or her mouth while wearing the mask, assumed in the awakening effort motion estimation device 1.

The distance calculating unit 105 outputs the reference point distance information to the motion estimating unit 106.

On the basis of the reference point distance information output from the distance calculating unit 105 in step ST5, the motion estimating unit 106 estimates whether or not the driver is performing the awakening effort motion depending on whether or not the reference point distance calculated by the distance calculating unit 105 satisfies the awakening effort estimating condition (step ST6).

The motion estimating unit 106 outputs the awakening effort estimating result to the awakening level decrease state estimating unit 107.

The awakening level decrease state estimating unit 107 estimates the awakening level decrease state of the driver in consideration of the awakening effort estimating result on the basis of the awakening effort estimating result output from the motion estimating unit 106 in step ST6 (step ST7).

The awakening level decrease state estimating unit 107 outputs the awakening level decrease state information to the output unit 108.

The output unit 108 outputs the awakening level decrease state information output from the awakening level decrease state estimating unit 107 to a device outside the awakening effort motion estimation device 1.

Note that, in the flowchart of FIG. 3, processing is performed in the order of step ST2 and step ST3, but this is merely an example, and the order of the processing of step ST2 and the processing of step ST3 may be reversed. In addition, the processing of step ST2 and the processing of step ST3 may be performed in parallel.

In addition, in the first embodiment, in a case where the awakening effort motion estimation device 1 does not include the face detecting unit 102 and the mask detecting unit 103, the processing of steps ST2 and ST3 can be omitted for the operation of the awakening effort motion estimation device 1 described above.

In addition, in the first embodiment, in a case where the awakening effort motion estimation device 1 does not include the awakening level decrease state estimating unit 107 and the output unit 108, the processing of steps ST7 and ST8 can be omitted for the operation of the awakening effort motion estimation device 1 described above.

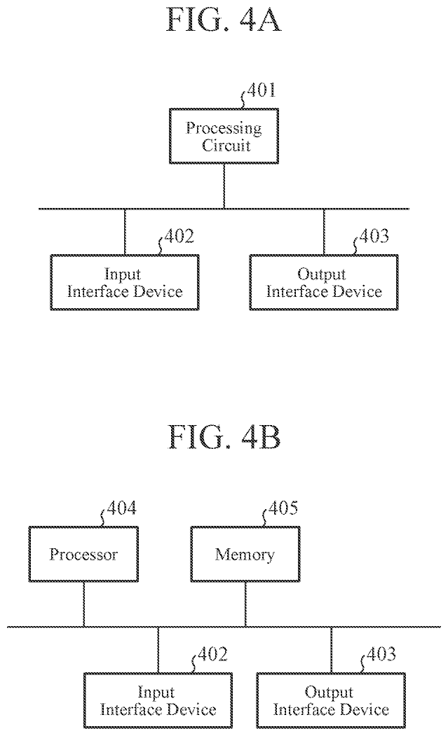

FIGS. 4A and 4B are each a diagram illustrating an example of a hardware configuration of the awakening effort motion estimation device 1 according to the first embodiment.

In the first embodiment, functions of the captured image acquiring unit 101, the face detecting unit 102, the mask detecting unit 103, the reference point detecting unit 104, the distance calculating unit 105, the motion estimating unit 106, the awakening level decrease state estimating unit 107, and the output unit 108 are implemented by a processing circuit 401. That is, the awakening effort motion estimation device 1 includes the processing circuit 401 for performing control to estimate whether or not an occupant wearing a mask is performing an awakening effort motion by moving his or her mouth on the basis of the captured image acquired from the camera 2.

The processing circuit 401 may be dedicated hardware as illustrated in FIG. 4A, or a processor 404 that executes a program stored in a memory as illustrated in FIG. 4B.

In a case where the processing circuit 401 is dedicated hardware, for example, a single circuit, a composite circuit, a programmed processor, a parallel programmed processor, an application specific integrated circuit (ASIC), a field-programmable gate array (FPGA), or a combination thereof corresponds to the processing circuit 401.

In a case where the processing circuit is the processor 404, the functions of the captured image acquiring unit 101, the face detecting unit 102, the mask detecting unit 103, the reference point detecting unit 104, the distance calculating unit 105, the motion estimating unit 106, the awakening level decrease state estimating unit 107, and the output unit 108 are implemented by software, firmware, or a combination of software and firmware. Software or firmware is described as a program and stored in a memory 405. The processor 404 executes the functions of the captured image acquiring unit 101, the face detecting unit 102, the mask detecting unit 103, the reference point detecting unit 104, the distance calculating unit 105, the motion estimating unit 106, the awakening level decrease state estimating unit 107, and the output unit 108 by reading and executing the program stored in the memory 405. That is, the awakening effort motion estimation device 1 includes the memory 405 for storing a program that causes steps ST1 to ST8 illustrated in FIG. 3 described above to be executed as a result when the program is executed by the processor 404. In addition, it can be said that the program stored in the memory 405 causes a computer to execute procedures or methods of processing performed by the captured image acquiring unit 101, the face detecting unit 102, the mask detecting unit 103, the reference point detecting unit 104, the distance calculating unit 105, the motion estimating unit 106, the awakening level decrease state estimating unit 107, and the output unit 108. Here, for example, a nonvolatile or volatile semiconductor memory such as RAM, read only memory (ROM), flash memory, erasable programmable read only memory (EPROM), or electrically erasable programmable read-only memory (EEPROM), a magnetic disk, a flexible disk, an optical disc, a compact disc, a mini disc, or a digital versatile disc (DVD) corresponds to the memory 405.

Note that some of the functions of the captured image acquiring unit 101, the face detecting unit 102, the mask detecting unit 103, the reference point detecting unit 104, the distance calculating unit 105, the motion estimating unit 106, the awakening level decrease state estimating unit 107, and the output unit 108 may be implemented by dedicated hardware, and some of the functions may be implemented by software or firmware. For example, the functions of the captured image acquiring unit 101 and the output unit 108 can be implemented by the processing circuit 401 as dedicated hardware, and the functions of the face detecting unit 102, the mask detecting unit 103, the reference point detecting unit 104, the distance calculating unit 105, the motion estimating unit 106, and the awakening level decrease state estimating unit 107 can be implemented by the processor 404 reading and executing a program stored in the memory 405.

The awakening effort motion estimation device 1 includes an input interface device 402 and an output interface device 403 that perform wired communication or wireless communication with a device such as the camera 2.

In the first embodiment described above, the awakening effort motion estimation device 1 is an in-vehicle device mounted on the vehicle 3, and the captured image acquiring unit 101, the face detecting unit 102, the mask detecting unit 103, the reference point detecting unit 104, the distance calculating unit 105, the motion estimating unit 106, the awakening level decrease state estimating unit 107, and the output unit 108 are included in the awakening effort motion estimation device 1.

It is not limited to this, and some of the captured image acquiring unit 101, the face detecting unit 102, the mask detecting unit 103, the reference point detecting unit 104, the distance calculating unit 105, the motion estimating unit 106, the awakening level decrease state estimating unit 107, and the output unit 108 may be mounted on an in-vehicle device of a vehicle, and the others may be included in a server connected to the in-vehicle device via a network. In this manner, the in-vehicle device and the server may constitute an awakening effort motion estimating system.

In addition, all of the captured image acquiring unit 101, the face detecting unit 102, the mask detecting unit 103, the reference point detecting unit 104, the distance calculating unit 105, the motion estimating unit 106, the awakening level decrease state estimating unit 107, and the output unit 108 may be included in the server.

As described above, according to the first embodiment, the awakening effort motion estimation device 1 includes: the captured image acquiring unit 101 that acquires a captured image obtained by imaging an occupant's face in a vehicle; the reference point detecting unit 104 that detects, on the basis of the captured image acquired by the captured image acquiring unit 101, in the captured image, two reference points for estimating a motion of the occupant's mouth, the two reference points being a point on a mask worn by the occupant, the other point being a point based on a feature point of the occupant's face or a point different from the one point on the mask; the distance calculating unit 105 that calculates a reference point distance between the two reference points detected by the reference point detecting unit 104; and the motion estimating unit 106 that estimates whether or not the occupant is performing an awakening effort motion by moving his or her mouth depending on whether or not the reference point distance calculated by the distance calculating unit 105 satisfies an awakening effort estimating condition. Therefore, the awakening effort motion estimation device 1 can estimate that an occupant is performing an awakening effort motion by moving his or her mouth even when the occupant wears a mask.

In addition, the awakening effort motion estimation device 1 can estimate that an occupant in the vehicle 3 is performing an awakening effort motion by moving his or her mouth in a case where the occupant wears a mask using the conventional camera 2 disposed for monitoring the occupant. That is, the awakening effort motion estimation device 1 can estimate whether or not an occupant is performing an awakening effort motion by moving his or her mouth in a case where the occupant wears a mask without requiring a new sensor or the like other than the camera 2.

### Second Embodiment

In the first embodiment, the awakening effort motion estimation device does not consider a direction of an occupant's face when estimating whether or not the occupant in the vehicle is performing an awakening effort motion.

In a second embodiment, an embodiment will be described in which an awakening effort motion estimation device estimates whether or not an occupant is performing an awakening effort motion in consideration of a direction of the occupant's face.

Note that, in the following second embodiment, the occupant is assumed to be a driver as in the first embodiment. However, this is merely an example, and the awakening effort motion estimation device can also estimate whether or not an occupant other than the driver is performing an awakening effort motion by moving his or her mouth.

In addition, the awakening effort motion estimation device according to the second embodiment is mounted on a vehicle similarly to the awakening effort motion estimation device according to the first embodiment. In addition, the awakening effort motion estimation device according to the second embodiment is connected to a camera mounted on the vehicle similarly to the awakening effort motion estimation device according to the first embodiment.

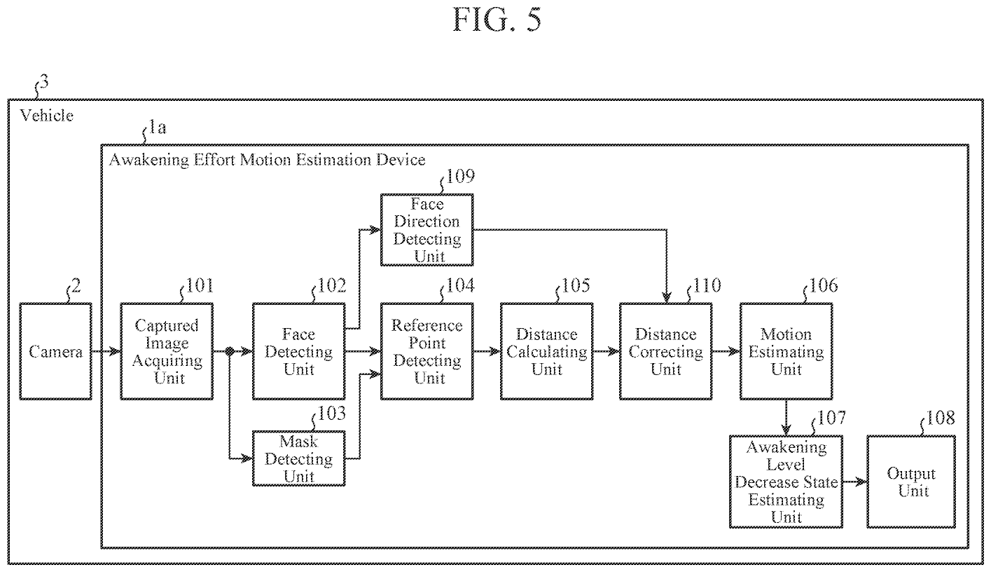

FIG. 5 is a diagram illustrating a configuration example of an awakening effort motion estimation device 1a according to the second embodiment.

In the configuration of the awakening effort motion estimation device 1a according to the second embodiment, the same components as those of the awakening effort motion estimation device 1 described with reference to FIG. 1 in the first embodiment are denoted by the same reference numerals, and redundant description is omitted.

Note that, similarly to the awakening effort motion estimation device 1 according to the first embodiment, the awakening effort motion estimation device 1a does not necessarily include a face detecting unit 102, a mask detecting unit 103, an awakening level decrease state estimating unit 107, and an output unit 108.

The awakening effort motion estimation device 1a according to the second embodiment is different from the awakening effort motion estimation device 1 according to the first embodiment in that the awakening effort motion estimation device 1a includes a face direction detecting unit 109 and a distance correcting unit 110.

The face direction detecting unit 109 detects a direction of a driver's face on the basis of the captured image acquired by a captured image acquiring unit 101.

More specifically, the face direction detecting unit 109 detects the direction of the driver's face on the basis of a captured image with a facial feature point to which a feature point of the driver's face detected by the face detecting unit 102 on the basis of the captured image acquired by the captured image acquiring unit 101 is imparted.

In the second embodiment, the face detecting unit 102 outputs the captured image with a facial feature point to the reference point detecting unit 104 and the face direction detecting unit 109.

Note that, for example, in a case where the face detecting unit 102 is included in a camera 2, the face direction detecting unit 109 only needs to acquire the captured image with a facial feature point from the camera 2 via the captured image acquiring unit 101.

For example, the face direction detecting unit 109 detects the direction of the driver's face using a face direction detector that has learned in advance, as training data, data obtained by imparting the direction of the driver's face as a teacher label to a large amount of face image data obtained by imaging faces in various directions.

The face direction detecting unit 109 obtains the direction of the driver's face by inputting the captured image with a facial feature point to the face direction detector.

For example, the face direction detecting unit 109 may obtain the direction of the driver's face by acquiring a captured image from the captured image acquiring unit 101 and inputting the captured image to a face direction detector.

In addition, for example, the face direction detecting unit 109 may detect the direction of the driver's face from a change in the position of a feature point of the driver's face in the captured image with the facial feature point on the basis of the captured image with the facial feature point. The face direction detecting unit 109 only needs to detect the direction of the driver's face on the basis of the face feature point captured image by various known methods.

In the second embodiment, the direction of the driver's face is assumed to be a vertical direction of the driver in a real space, in other words, a face direction in a pitch direction. The direction of the driver's face is represented by, for example, a pitch angle.

For example, in the second embodiment, the direction of the driver's face in a case where the driver's face is directed right in front of the camera 2 is defined as 0 degrees, the direction of the driver's face is positive when the driver's face is directed upward, and the direction of the driver's face is negative when the driver's face is directed downward.

Note that this is merely an example, and for example, in a case where the direction of the driver's face in the pitch direction when the driver faces in a windshield direction is defined as 0 degrees in the PMS specification, the face direction detecting unit 109 defines the direction of the driver's face in a case where the driver's face is directed right in front of the windshield as 0 degrees, and considers an offset value based on a difference in height between the position of one point of the windshield that is in front of the driver's face and the position where the camera 2 is disposed.

The face direction detecting unit 109 outputs information regarding the detected direction of the driver's face to the distance correcting unit 110.

The distance correcting unit 110 corrects the reference point distance calculated by the distance calculating unit 105 on the basis of the direction of the driver's face detected by the face direction detecting unit 109.

In the second embodiment, the distance calculating unit 105 outputs the reference point distance information to the distance correcting unit 110.

For example, the distance correcting unit 110 corrects the reference point distance in a case where the direction of the driver's face detected by the face direction detecting unit 109 is not the front, and does not correct the reference point distance in a case where the direction of the driver's face detected by the face direction detecting unit 109 is the front. The state in which the direction of the driver's face is the front is, for example, a state in which the direction of the driver's face detected by the face direction detecting unit 109 is within a range of −5 degrees to 5 degrees.

The distance correcting unit 110 corrects the reference point distance using, for example, the following (Formula 1). θ=direction of driver's face.

Corrected reference point distance=Reference point distance before correction×cos θ (Formula 1)

Note that the distance correcting unit 110 corrects the reference point distances in all the patterns on the basis of the direction of the driver's face detected by the face direction detecting unit 109.

The distance correcting unit 110 updates the reference point distance to the corrected reference point distance in the reference point distance information output from the distance calculating unit 105, and outputs the reference point distance information regarding the updated reference point distance to the motion estimating unit 106. In a case where the distance correcting unit 110 has not corrected the reference point distance, the distance correcting unit 110 outputs the reference point distance information output from the distance calculating unit 105 to the motion estimating unit 106.

In the second embodiment, on the basis of the reference point distance information output from the distance correcting unit 110, the motion estimating unit 106 estimates whether or not the driver is performing the awakening effort motion depending on whether or not the reference point distance calculated by the distance calculating unit 105 satisfies the awakening effort estimating condition. When the reference point distance calculated by the distance calculating unit 105 has been corrected by the distance correcting unit 110, the motion estimating unit 106 estimates whether or not the driver is performing the awakening effort motion depending on whether or not the corrected reference point distance satisfies the awakening effort estimating condition.

An operation of the awakening effort motion estimation device 1a according to the second embodiment will be described.

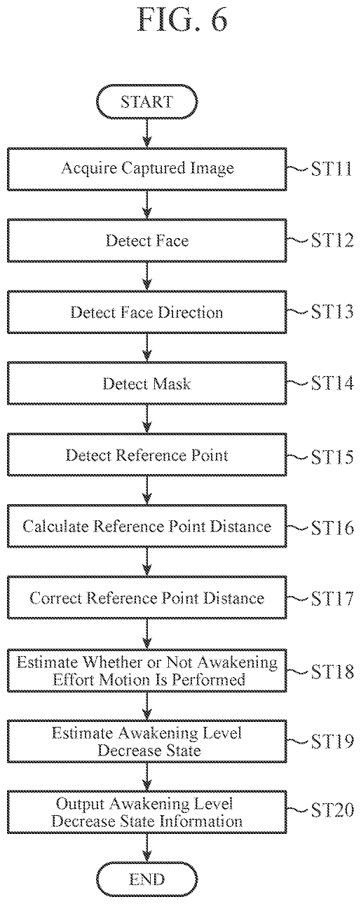

FIG. 6 is a flowchart for describing the operation of the awakening effort motion estimation device 1a according to the second embodiment.

The awakening effort motion estimation device 1a repeats the operation illustrated in the flowchart of FIG. 6, for example, from when the engine of a vehicle 3 is turned on until the engine is turned off.

Since specific operations in steps ST11, ST12, ST14 to ST16, ST18, and ST20 in FIG. 6 are similar to those in steps ST1 to ST8 in FIG. 3 described in the first embodiment, respectively, redundant description is omitted.

The face direction detecting unit 109 detects the direction of the driver's face on the basis of the captured image acquired by the captured image acquiring unit 101 in step ST11.

More specifically, the face direction detecting unit 109 detects the direction of the driver's face on the basis of a captured image with a facial feature point to which a feature point of the driver's face detected by the face detecting unit 102 on the basis of the captured image acquired by the captured image acquiring unit 101 in step ST12 is imparted (step ST13).

The face direction detecting unit 109 outputs information regarding the detected direction of the driver's face to the distance correcting unit 110.

The distance correcting unit 110 corrects the reference point distance calculated by the distance calculating unit 105 in step ST16 on the basis of the direction of the driver's face detected by the face direction detecting unit 109 in step ST13 (step ST17).

The distance correcting unit 110 updates the reference point distance to the corrected reference point distance in the reference point distance information output from the distance calculating unit 105, and outputs the reference point distance information regarding the updated reference point distance to the motion estimating unit 106.

Note that, in a case where the distance correcting unit 110 has not corrected the reference point distance, the distance correcting unit 110 outputs the reference point distance information output from the distance calculating unit 105 to the motion estimating unit 106.

On the basis of the reference point distance information output from the distance correcting unit 110 in step ST17, the motion estimating unit 106 estimates whether or not the driver is performing the awakening effort motion depending on whether or not the reference point distance calculated by the distance calculating unit 105 in step ST16 satisfies the awakening effort estimating condition (step ST18).

Note that, in the flowchart of FIG. 6, processing is performed in the order of step ST12, step ST13, and step ST14, but this is merely an example, and the order of the processing of steps ST12 and ST13 and the processing of step ST14 may be reversed. In addition, the processing of steps ST12 and ST13 and the processing of step ST14 may be performed in parallel.

In addition, in the second embodiment, in a case where the awakening effort motion estimation device 1a does not include the face detecting unit 102 and the mask detecting unit 103, the processing of steps ST12 and ST14 can be omitted for the operation of the awakening effort motion estimation device 1a described above.

In addition, in the second embodiment, in a case where the awakening effort motion estimation device 1a does not include the awakening level decrease state estimating unit 107 and the output unit 108, the processing of steps ST19 and ST20 can be omitted for the operation of the awakening effort motion estimation device 1a described above.

For example, when the direction of the driver's face changes in the vertical direction, in other words, the pitch direction, the reference point distance on the captured image changes even in a case where the driver's mouth is not moving. When the reference point distance changes, the awakening effort motion estimation device 1 according to the first embodiment assumes that the driver moves his or her mouth, and estimates whether or not the driver is performing the awakening effort motion by moving his or her mouth on the basis of the reference point distance.

Meanwhile, as described above, in a case where the direction of the driver's face changes in the vertical direction, the awakening effort motion estimation device 1a according to the second embodiment corrects the reference point distance in response to the change, and estimates whether or not the driver is performing the awakening effort motion on the basis of the corrected reference point distance. As a result, the awakening effort motion estimation device 1a can reduce erroneous estimation of whether or not the driver is performing the awakening effort motion and improve estimation accuracy of whether or not the driver is performing the awakening effort motion as compared with a case where the direction of the driver's face is not considered.

Since a hardware configuration of the awakening effort motion estimation device 1a according to the second embodiment is similar to the hardware configuration of the awakening effort motion estimation device 1 described with reference to FIGS. 4A and 4B in the first embodiment, description thereof is omitted.

In the second embodiment, functions of the captured image acquiring unit 101, the face detecting unit 102, the mask detecting unit 103, the reference point detecting unit 104, the distance calculating unit 105, the motion estimating unit 106, the awakening level decrease state estimating unit 107, the output unit 108, the face direction detecting unit 109, and the distance correcting unit 110 are implemented by the processing circuit 401. That is, the awakening effort motion estimation device 1a includes the processing circuit 401 for performing control to estimate whether or not an occupant wearing a mask is performing an awakening effort motion by moving his or her mouth, in consideration of a direction of the occupant's face on the basis of the captured image acquired from the camera 2.

The processing circuit 401 executes the functions of the captured image acquiring unit 101, the face detecting unit 102, the mask detecting unit 103, the reference point detecting unit 104, the distance calculating unit 105, the motion estimating unit 106, the awakening level decrease state estimating unit 107, the output unit 108, the face direction detecting unit 109, and the distance correcting unit 110 by reading and executing a program stored in the memory 405. That is, the awakening effort motion estimation device 1a includes the memory 405 for storing a program that causes steps ST11 to ST20 illustrated in FIG. 6 described above to be executed as a result when the program is executed by the processing circuit 401. In addition, it can be said that the program stored in the memory 405 causes a computer to execute procedures or methods performed by the captured image acquiring unit 101, the face detecting unit 102, the mask detecting unit 103, the reference point detecting unit 104, the distance calculating unit 105, the motion estimating unit 106, the awakening level decrease state estimating unit 107, the output unit 108, the face direction detecting unit 109, and the distance correcting unit 110.

The awakening effort motion estimation device 1a includes the input interface device 402 and the output interface device 403 that perform wired communication or wireless communication with a device such as the camera 2.

In the second embodiment described above, the awakening effort motion estimation device 1a is an in-vehicle device mounted on the vehicle 3, and the captured image acquiring unit 101, the face detecting unit 102, the mask detecting unit 103, the reference point detecting unit 104, the distance calculating unit 105, the motion estimating unit 106, the awakening level decrease state estimating unit 107, the output unit 108, the face direction detecting unit 109, and the distance correcting unit 110 are included in the awakening effort motion estimation device 1a.

It is not limited to this, and some of the captured image acquiring unit 101, the face detecting unit 102, the mask detecting unit 103, the reference point detecting unit 104, the distance calculating unit 105, the motion estimating unit 106, the awakening level decrease state estimating unit 107, the output unit 108, the face direction detecting unit 109, and the distance correcting unit 110 may be mounted on an in-vehicle device of a vehicle, and the others may be included in a server connected to the in-vehicle device via a network. In this manner, the in-vehicle device and the server may constitute an awakening effort motion estimating system.

In addition, all of the captured image acquiring unit 101, the face detecting unit 102, the mask detecting unit 103, the reference point detecting unit 104, the distance calculating unit 105, the motion estimating unit 106, the awakening level decrease state estimating unit 107, the output unit 108, the face direction detecting unit 109, and the distance correcting unit 110 may be included in the server.

As described above, according to the second embodiment, the awakening effort motion estimation device 1a includes: the face direction detecting unit 109 that detects a direction of an occupant's face on the basis of a captured image acquired by the captured image acquiring unit 101; and the distance correcting unit 110 that corrects a reference point distance calculated by the distance calculating unit 105 on the basis of the direction of the occupant's face detected by the face direction detecting unit 109, in which the motion estimating unit 106 estimates whether or not the occupant is performing an awakening effort motion by moving his or her mouth depending on whether or not the reference point distance corrected by the distance correcting unit 110 satisfies the awakening effort estimating condition. Therefore, the awakening effort motion estimation device 1a can estimate that an occupant is performing an awakening effort motion by moving his or her mouth in consideration of a direction of the occupant's face.

### Third Embodiment

In the first embodiment, the awakening effort motion estimation device does not consider the size of a mask (hereinafter, referred to as “mask size”) worn by an occupant in the vehicle when estimating whether or not the occupant is performing an awakening effort motion by moving his or her mouth.

In a third embodiment, an embodiment will be described in which an awakening effort motion estimation device estimates whether or not an occupant is performing an awakening effort motion by moving his or her mouth in consideration of a mask size worn by the occupant.

Note that, in the following third embodiment, the occupant is assumed to be a driver as in the first embodiment. However, this is merely an example, and the awakening effort motion estimation device can also estimate whether or not an occupant other than the driver is performing an awakening effort motion by moving his or her mouth.

In addition, the awakening effort motion estimation device according to the third embodiment is mounted on a vehicle similarly to the awakening effort motion estimation device according to the first embodiment. In addition, the awakening effort motion estimation device according to the third embodiment is connected to a camera mounted on the vehicle similarly to the awakening effort motion estimation device according to the first embodiment.

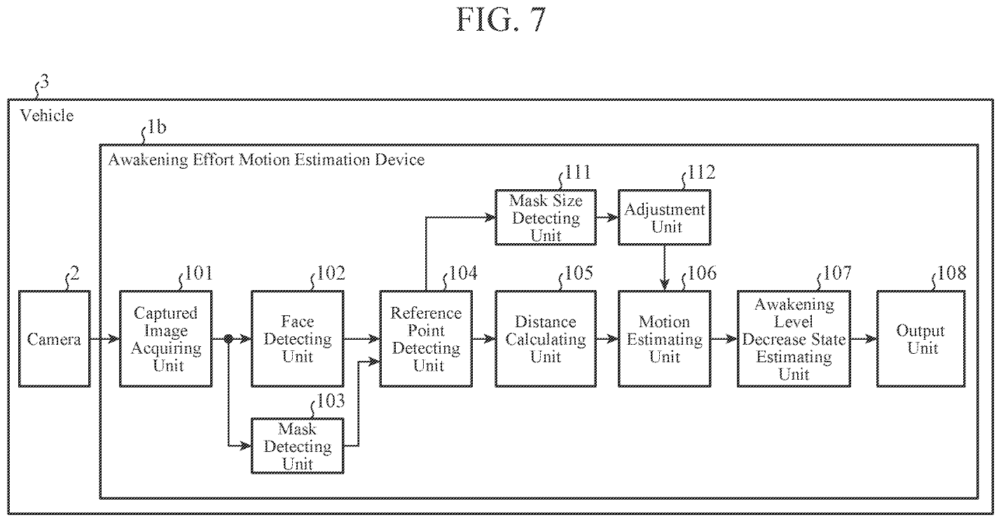

FIG. 7 is a diagram illustrating a configuration example of an awakening effort motion estimation device 1b according to the third embodiment.

In the configuration of the awakening effort motion estimation device 1b according to the third embodiment, the same components as those of the awakening effort motion estimation device 1 described with reference to FIG. 1 in the first embodiment are denoted by the same reference numerals, and redundant description is omitted.

Note that, similarly to the awakening effort motion estimation device 1 according to the first embodiment, the awakening effort motion estimation device 1b does not necessarily include a face detecting unit 102, a mask detecting unit 103, an awakening level decrease state estimating unit 107, and an output unit 108.

The awakening effort motion estimation device 1b according to the third embodiment is different from the awakening effort motion estimation device 1 according to the first embodiment in that the awakening effort motion estimation device 1b includes a mask size detecting unit 111 and an adjustment unit 112.

The mask size detecting unit 111 detects a mask size worn by a driver. Specifically, the mask size detecting unit 111 detects, as the mask size, the size of a mask worn by the driver in a longitudinal direction from an uppermost point of an upper end of the mask and a lowermost point of a lower end of the mask on the basis of a captured image after reference point impartment output from the reference point detecting unit 104. Note that, in the third embodiment, the mask size detected by the mask size detecting unit 111 is the mask size in a captured image.

For example, after the camera 2 is activated, in other words, after the awakening effort motion estimation device 1b is activated and the captured image acquiring unit 101 starts to acquire a captured image from the camera 2, the mask size detecting unit 111 calculates a mask size on the basis of the captured image after reference point impartment in units of frames for the first few minutes (for example, one minute), and detects a mode value, a median value, or an average value of the detected mask sizes as the mask size. For example, the reference point detecting unit 104 stores the captured image after reference point impartment in a storage unit, and the mask size detecting unit 111 refers to the storage unit and acquires the captured image after reference point impartment for the first few minutes after the camera 2 is activated.

In addition, for example, the mask size detecting unit 111 may detect the mask size on the basis of a captured image after reference point impartment of a certain one frame.

The mask size detecting unit 111 outputs information regarding the detected mask size to the adjustment unit 112.

The adjustment unit 112 calculates the size of the driver's face from a feature point of the driver's face based on the captured image acquired by the captured image acquiring unit 101, and adjusts a yawning determination threshold on the basis of the calculated size of the driver's face and the mask size detected by the mask size detecting unit 111.

In the third embodiment, specific contents of the awakening effort estimating condition are <condition 1> to <condition 5> of the awakening effort estimating condition described in the first embodiment. In this case, the yawning determination threshold is the distance determination threshold of <condition 1> of the awakening effort estimating condition described in the first embodiment.

For example, the adjustment unit 112 adjusts the distance determination threshold by the following method.

First, the adjustment unit 112 calculates a mask size appropriate for the size of the driver's face (hereinafter, referred to as “optimum mask size”) on the basis of the captured image after reference point impartment. Note that the adjustment unit 112 only needs to acquire the captured image after reference point impartment via the mask size detecting unit 111.

For example, the adjustment unit 112 calculates the size of the driver's face, and defines a value obtained by multiplying the calculated size of the driver's face by a predetermined value (hereinafter, referred to as “optimum size calculating value”) as an optimal mask size of the driver. The optimum size calculating value is, for example, “0.5”. The adjustment unit 112 calculates the size of the driver's face from a feature point of the driver's face on the basis of the captured image after reference point impartment. Specifically, for example, the adjustment unit 112 calculates the size of the driver's face from a feature point indicating the top of the head of the driver and a feature point indicating the jaw. For example, the adjustment unit 112 may calculate a distance from a feature point indicating a lowermost part of the driver's eye to a feature point indicating the jaw as the size of the driver's face. Note that it is assumed that the feature point indicating the lowermost part of the driver's eye has been detected by the face detecting unit 102.

After calculating the optimum mask size of the driver, the adjustment unit 112 calculates an adjusted yawning determination threshold using, for example, the following (Formula 2).

Adjusted yawning determination threshold=Yawning determination threshold×Optimum mask size/Actual mask size (Formula 2)

In (Formula 2), the actual mask size is a mask size detected by the mask size detecting unit 111.

The adjustment unit 112 outputs the calculated adjusted yawning determination threshold to the motion estimating unit 106.

In the third embodiment, the motion estimating unit 106 estimates whether or not the driver is performing an awakening effort motion using the yawning determination threshold for estimating that the driver is performing the awakening effort motion, which is set in the awakening effort motion estimating condition, as the yawning determination threshold adjusted by the adjustment unit 112.

An operation of the awakening effort motion estimation device 1b according to the third embodiment will be described.

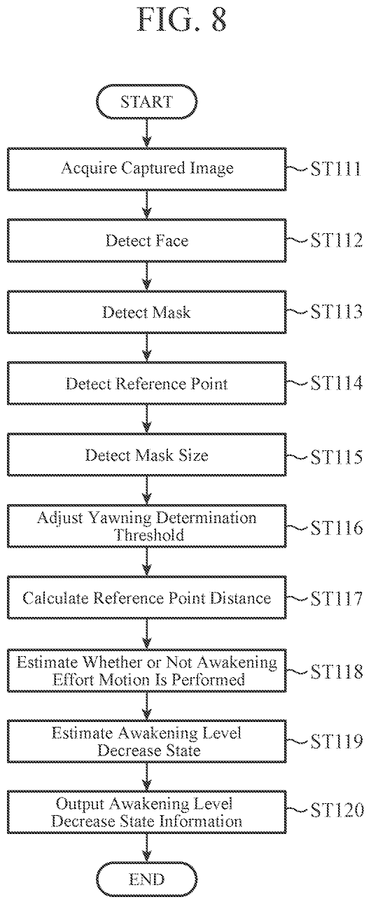

FIG. 8 is a flowchart for describing the operation of the awakening effort motion estimation device 1b according to the third embodiment.

The awakening effort motion estimation device 1b repeats the operation illustrated in the flowchart of FIG. 8, for example, from when the engine of a vehicle 3 is turned on until the engine is turned off.

Since specific operations in steps ST111 to ST114, ST117, ST119, and ST120 in FIG. 8 are similar to those in steps ST1 to ST5, ST7, and ST8 in FIG. 3 described in the first embodiment, respectively, redundant description is omitted.

The mask size detecting unit 111 detects a mask size worn by a driver. Specifically, the mask size detecting unit 111 detects, as the mask size, the size of a mask worn by the driver in a longitudinal direction from an uppermost point of an upper end of the mask and a lowermost point of a lower end of the mask on the basis of a captured image after reference point impartment output from the reference point detecting unit 104 in step ST114 (step ST115).

The mask size detecting unit 111 outputs information regarding the detected mask size to the adjustment unit 112.

The adjustment unit 112 calculates the size of the driver's face from a feature point of the driver's face based on the captured image acquired by the captured image acquiring unit 101 in step ST111, and adjusts a yawning determination threshold on the basis of the calculated size of the driver's face and the mask size detected by the mask size detecting unit 111 (step ST116).

The adjustment unit 112 outputs the calculated adjusted yawning determination threshold to the motion estimating unit 106.

In step ST118, the motion estimating unit 106 estimates whether or not the driver is performing an awakening effort motion using a yawning determination threshold for estimating that the driver is performing the awakening effort motion by yawning, which is set in the awakening effort motion estimating condition, as the yawning determination threshold adjusted by the adjustment unit 112 in step ST116.

Note that, in the flowchart of FIG. 8, processing is performed in the order of step ST112 and step ST113, but this is merely an example, and the order of the processing of step ST112 and the processing of step ST113 may be reversed. In addition, the processing of step ST112 and the processing of step ST113 may be performed in parallel.

In the flowchart of FIG. 8, the processing of step ST116 is performed before the processing of step ST117, but this is merely an example, and the processing of step ST116 only needs to be performed before the processing of step ST118 is performed.

In addition, in the third embodiment, in a case where the awakening effort motion estimation device 1b does not include the face detecting unit 102 and the mask detecting unit 103, the processing of steps ST112 and ST113 can be omitted for the operation of the awakening effort motion estimation device 1b described above.

In addition, in the third embodiment, in a case where the awakening effort motion estimation device 1b does not include the awakening level decrease state estimating unit 107 and the output unit 108, the processing of steps ST119 and ST120 can be omitted for the operation of the awakening effort motion estimation device 1b described above.

For example, in a case where the mask size is larger than the size of the driver's face, the moving amount of the mask when the driver moves his or her mouth is smaller than that in a case where the driver wears a mask matching the size of his or her face. Therefore, when the awakening effort motion estimation device 1b estimates whether or not the driver is performing the awakening effort motion without considering the mask size, there is a possibility that the awakening effort motion estimation device 1b cannot accurately estimate that the driver is performing the awakening effort motion.

Meanwhile, as described above, the awakening effort motion estimation device 1b according to the third embodiment detects the mask size worn by the driver, and adjusts the yawning determination threshold in the awakening effort estimating condition for estimating the awakening effort motion by yawning of the driver on the basis of the size of the driver's face and the mask size. Then, the awakening effort motion estimation device 1b estimates whether or not the driver is performing the awakening effort motion using the yawning determination threshold set in the awakening effort motion estimating condition as the adjusted yawning determination threshold.

As a result, the awakening effort motion estimation device 1b can reduce erroneous estimation of whether or not the driver is performing the awakening effort motion and improve estimation accuracy as compared with a case where the mask size worn by the driver is not considered.

Since a hardware configuration of the awakening effort motion estimation device 1b according to the third embodiment is similar to the hardware configuration of the awakening effort motion estimation device 1 described with reference to FIGS. 4A and 4B in the first embodiment, description thereof is omitted.

In the third embodiment, functions of the captured image acquiring unit 101, the face detecting unit 102, the mask detecting unit 103, the reference point detecting unit 104, the distance calculating unit 105, the motion estimating unit 106, the awakening level decrease state estimating unit 107, the output unit 108, the mask size detecting unit 111, and the adjustment unit 112 are implemented by the processing circuit 401. That is, the awakening effort motion estimation device 1b includes the processing circuit 401 for performing control to estimate whether or not an occupant is performing an awakening effort motion by moving his or her mouth, in consideration of a mask size of the occupant on the basis of the captured image acquired from the camera 2.

The processing circuit 401 executes the functions of the captured image acquiring unit 101, the face detecting unit 102, the mask detecting unit 103, the reference point detecting unit 104, the distance calculating unit 105, the motion estimating unit 106, the awakening level decrease state estimating unit 107, the output unit 108, the mask size detecting unit 111, and the adjustment unit 112 by reading and executing a program stored in the memory 405. That is, the awakening effort motion estimation device 1b includes the memory 405 for storing a program that causes steps ST111 to ST120 illustrated in FIG. 8 described above to be executed as a result when the program is executed by the processing circuit 401. In addition, it can be said that the program stored in the memory 405 causes a computer to execute procedures or methods performed by the captured image acquiring unit 101, the face detecting unit 102, the mask detecting unit 103, the reference point detecting unit 104, the distance calculating unit 105, the motion estimating unit 106, the awakening level decrease state estimating unit 107, the output unit 108, the mask size detecting unit 111, and the adjustment unit 112.

The awakening effort motion estimation device 1b includes the input interface device 402 and the output interface device 403 that perform wired communication or wireless communication with a device such as the camera 2.

In the third embodiment described above, the awakening effort motion estimation device 1b is an in-vehicle device mounted on the vehicle 3, and the captured image acquiring unit 101, the face detecting unit 102, the mask detecting unit 103, the reference point detecting unit 104, the distance calculating unit 105, the motion estimating unit 106, the awakening level decrease state estimating unit 107, the output unit 108, the mask size detecting unit 111, and the adjustment unit 112 are included in the awakening effort motion estimation device 1b.

It is not limited to this, and some of the captured image acquiring unit 101, the face detecting unit 102, the mask detecting unit 103, the reference point detecting unit 104, the distance calculating unit 105, the motion estimating unit 106, the awakening level decrease state estimating unit 107, the output unit 108, the mask size detecting unit 111, and the adjustment unit 112 may be mounted on an in-vehicle device of a vehicle, and the others may be included in a server connected to the in-vehicle device via a network. In this manner, the in-vehicle device and the server may constitute an awakening effort motion estimating system.

In addition, all of the captured image acquiring unit 101, the face detecting unit 102, the mask detecting unit 103, the reference point detecting unit 104, the distance calculating unit 105, the motion estimating unit 106, the awakening level decrease state estimating unit 107, the output unit 108, the mask size detecting unit 111, and the adjustment unit 112 may be included in the server.

As described above, according to the third embodiment, the awakening effort motion estimation device 1b includes: the mask size detecting unit 111 that detects the size of a mask on the basis of a distance between one point of an upper end of the mask and one point of a lower end of the mask; and the adjustment unit 112 that calculates the size of the occupant's face from a feature point of the occupant's face based on a captured image acquired by the captured image acquiring unit 101 and adjusts a yawning determination threshold on the basis of the calculated size of the occupant's face and the size of the mask detected by the mask size detecting unit 111, in which the motion estimating unit 106 estimates that the occupant is performing an awakening effort motion by moving his or her mouth in a case where a state where the reference point distance is equal to or more than the yawning determination threshold adjusted by the adjustment unit 112 continues for equal to or more than the first determination time or in a case where the periodic change of the reference point distance continues for equal to or more than the second determination time and less than the third determination time longer than the second determination time. Therefore, the awakening effort motion estimation device 1b can estimate that an occupant is performing an awakening effort motion by moving his or her mouth in consideration of a mask size of the occupant.

### Fourth Embodiment

In the first embodiment, whether or not an occupant is performing an awakening effort motion is estimated by a motion of a mask in a captured image.

However, the mask also moves, for example, in a case where the occupant corrects an improper position of the mask with his or her hand, in a case where the occupant exposes his or her nose, lowering the mask with his or her hand (a state where the nose is not covered with the mask), or in a case where the occupant stops exposing his or her nose, using the mask with his or her hand. In a case where such a motion of correcting the position of the mask with the hand is performed by the occupant, the awakening effort motion estimation device may erroneously estimate the awakening effort motion of the occupant.

In a fourth embodiment, an embodiment will be described in which an awakening effort motion estimation device prevents erroneous estimation of the awakening effort motion of an occupant when the occupant performs a motion of correcting the position of a mask with his or her hand.

Note that, in the following fourth embodiment, the occupant is assumed to be a driver as in the first embodiment. However, this is merely an example, and the awakening effort motion estimation device can also estimate whether or not an occupant other than the driver is performing an awakening effort motion by moving his or her mouth.

In addition, the awakening effort motion estimation device according to the fourth embodiment is mounted on a vehicle similarly to the awakening effort motion estimation device according to the first embodiment. In addition, the awakening effort motion estimation device according to the fourth embodiment is connected to a camera mounted on the vehicle similarly to the awakening effort motion estimation device according to the first embodiment.

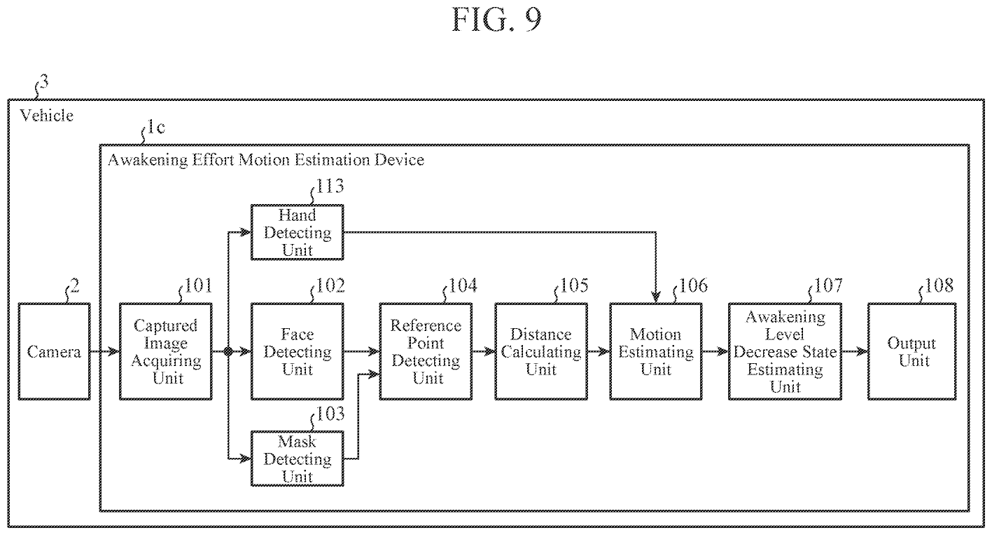

FIG. 9 is a diagram illustrating a configuration example of an awakening effort motion estimation device 1c according to the fourth embodiment.

In the configuration of the awakening effort motion estimation device 1c according to the fourth embodiment, the same components as those of the awakening effort motion estimation device 1 described with reference to FIG. 1 in the first embodiment are denoted by the same reference numerals, and redundant description is omitted.

Note that, similarly to the awakening effort motion estimation device 1 according to the first embodiment, the awakening effort motion estimation device 1c does not necessarily include a face detecting unit 102, a mask detecting unit 103, an awakening level decrease state estimating unit 107, and an output unit 108.

The awakening effort motion estimation device 1c according to the fourth embodiment is different from the awakening effort motion estimation device 1 according to the first embodiment in that the awakening effort motion estimation device 1c includes a hand detecting unit 113.

On the basis of a captured image acquired by a captured image acquiring unit 101, the hand detecting unit 113 detects whether or not a driver's hand is present within a detection range of the driver's face (hereinafter, referred to as “face detecting range”) in the captured image.

In the fourth embodiment, the face detecting range is a range in which a face is assumed to be detected on the captured image. Specifically, for example, the face detecting range is a range in which a space near the front of a headrest is imaged in the captured image. The face detecting range is set in advance depending on the position where the camera 2 is disposed.

Note that, in the fourth embodiment, the captured image acquiring unit 101 outputs a captured image to the face detecting unit 102, the mask detecting unit 103, and the hand detecting unit 113.

For example, the hand detecting unit 113 detects whether or not a driver's hand is present within the driver's face detecting range using a hand detector that has learned in advance, as training data, data obtained by imparting information of whether or not a hand is present within the face detecting range as a teacher label to a large amount of face image data in which various faces are imaged. Note that the face image data included in the training data includes face image data in which a hand is present within the driver's face detecting range.

The hand detecting unit 113 inputs a captured image acquired by the captured image acquiring unit 101 to the hand detector, and obtains information indicating whether or not the driver's hand is present within the driver's face detecting range.

In addition, the hand detecting unit 113 may detect whether or not the driver's hand is present within the driver's face detecting range from the captured image using a known image recognition technique such as pattern matching.

In addition, for example, in a case where PMS has a hand gesture detecting function, the hand detecting unit 113 may acquire a detection result of a hand gesture from PMS and detect whether or not the driver's hand is present within the driver's face detecting range.

The hand detecting unit 113 outputs information indicating whether or not a hand is present within the driver's face detecting range (hereinafter, referred to as “hand presence or absence information”) to the motion estimating unit 106.

In the fourth embodiment, in a case where the hand detecting unit 113 detects that the driver's hand is present within the driver's face detecting range, the motion estimating unit 106 does not estimate whether or not the driver is performing the awakening effort motion until a preset time (hereinafter, referred to as “estimated stop time”) elapses. Note that the motion estimating unit 106 can determine that the hand detecting unit 113 has detected that the driver's hand is present within the driver's face detecting range on the basis of the hand presence or absence information output from the hand detecting unit 113.

Specifically, for example, when the hand presence or absence information indicating that the driver's hand is present within the driver's face detecting range is output from the hand detecting unit 113, the motion estimating unit 106 starts counting the estimated stop time from the time when the hand presence or absence information is acquired. The motion estimating unit 106 stops processing of estimating whether or not the driver is performing the awakening effort motion until the estimated stop time elapses. When the motion estimating unit 106 stops the estimation processing, an awakening effort estimating result is not output to the awakening level decrease state estimating unit 107. Therefore, the awakening level decrease state estimating unit 107 also does not perform the processing of estimating the awakening level decrease state of the driver until the estimated stop time elapses. Until the estimated stop time elapses, awakening level decrease state information is not output from the output unit 108.

An operation of the awakening effort motion estimation device 1c according to the fourth embodiment will be described.

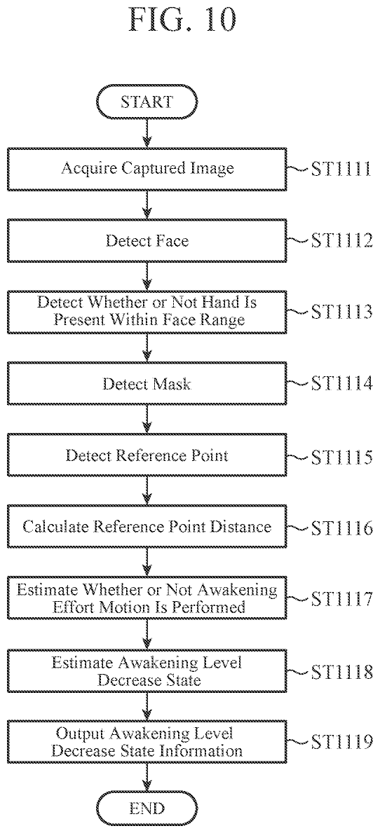

FIG. 10 is a flowchart for describing the operation of the awakening effort motion estimation device 1c according to the fourth embodiment.

The awakening effort motion estimation device 1c repeats the operation illustrated in the flowchart of FIG. 10, for example, from when the engine of a vehicle 3 is turned on until the engine is turned off.

Since steps ST1111, ST1112, and ST1114 to ST1119 in FIG. 10 are similar to specific operations in steps ST1 to ST8 in FIG. 3 described in the first embodiment, respectively, redundant description is omitted.

The hand detecting unit 113 detects whether or not the driver's hand is present within the driver's face detecting range on the basis of the captured image acquired by the captured image acquiring unit 101 in step ST1111 (step ST1113).

In step ST1117, in a case where the hand detecting unit 113 detects that the driver's hand is present within a range where the driver's face has been detected, the motion estimating unit 106 does not estimate whether or not the driver is performing the awakening effort motion until the estimated stop time elapses.

Since the awakening effort estimating result is not output from the motion estimating unit 106 until the estimated stop time elapses, the awakening level decrease state estimating unit 107 also does not estimate the awakening level decrease state of the driver until the estimated stop time elapses. Until the estimated stop time elapses, the awakening level decrease state information is not output from the output unit 108.

Note that, regarding the operation of the awakening effort motion estimation device 1c described with reference to FIG. 10, when the hand detecting unit 113 detects that the driver's hand is present within the driver's face detecting range in step ST1113, the operation of the awakening effort motion estimation device 1c may not perform the processing of steps ST1114 to ST1119 until the estimated stop time elapses.

In the awakening effort motion estimation device 1c, in a case where the hand detecting unit 113 detects that the driver's hand is present within the driver's face detecting range, it is only required for the motion estimating unit 106 not to estimate whether or not the driver is performing the awakening effort motion by moving his or her mouth until the estimated stop time elapses.

In addition, in the flowchart of FIG. 10, processing is performed in the order of step ST1112, step ST1113, and step ST1114, but this is merely an example, and the order of the processing of steps ST1112 to ST1114 does not matter. The processing of steps ST1112 to ST1114 may be performed in parallel.

In addition, in the fourth embodiment, in a case where the awakening effort motion estimation device 1c does not include the face detecting unit 102 and the mask detecting unit 103, the processing of steps ST1112 and ST1114 can be omitted for the operation of the awakening effort motion estimation device 1c described above.

In addition, in the fourth embodiment, in a case where the awakening effort motion estimation device 1c does not include the awakening level decrease state estimating unit 107 and the output unit 108, the processing of steps ST1118 and ST1119 can be omitted for the operation of the awakening effort motion estimation device 1c described above.

As described above, for example, in a case where the driver performs a motion of correcting the position of a mask with his or her hand, the awakening effort motion estimation device 1c temporarily stops the estimation of whether or not the driver is performing the awakening effort motion by moving his or her mouth. As a result, the awakening effort motion estimation device 1c can reduce erroneous estimation of the awakening effort motion by moving the driver's mouth, and improve estimation accuracy of the awakening effort motion.

Since a hardware configuration of the awakening effort motion estimation device 1c according to the fourth embodiment is similar to the hardware configuration of the awakening effort motion estimation device 1 described with reference to FIGS. 4A and 4B in the first embodiment, description thereof is omitted.

In the fourth embodiment, functions of the captured image acquiring unit 101, the face detecting unit 102, the mask detecting unit 103, the reference point detecting unit 104, the distance calculating unit 105, the motion estimating unit 106, the awakening level decrease state estimating unit 107, the output unit 108, and the hand detecting unit 113 are implemented by the processing circuit 401. That is, the awakening effort motion estimation device 1c includes the processing circuit 401 for performing control to estimate whether or not an occupant is performing an awakening effort motion by moving his or her mouth, in consideration of a mask size of the occupant on the basis of the captured image acquired from the camera 2.

The processing circuit 401 executes the functions of the captured image acquiring unit 101, the face detecting unit 102, the mask detecting unit 103, the reference point detecting unit 104, the distance calculating unit 105, the motion estimating unit 106, the awakening level decrease state estimating unit 107, the output unit 108, and the hand detecting unit 113 by reading and executing a program stored in the memory 405. That is, the awakening effort motion estimation device 1c includes the memory 405 for storing a program that causes steps ST1111 to ST1119 illustrated in FIG. 10 described above to be executed as a result when the program is executed by the processing circuit 401. In addition, it can be said that the program stored in the memory 405 causes a computer to execute procedures or methods performed by the captured image acquiring unit 101, the face detecting unit 102, the mask detecting unit 103, the reference point detecting unit 104, the distance calculating unit 105, the motion estimating unit 106, the awakening level decrease state estimating unit 107, the output unit 108, and the hand detecting unit 113.

The awakening effort motion estimation device 1c includes the input interface device 402 and the output interface device 403 that perform wired communication or wireless communication with a device such as the camera 2.

In the fourth embodiment described above, the awakening effort motion estimation device 1c is an in-vehicle device mounted on the vehicle 3, and the captured image acquiring unit 101, the face detecting unit 102, the mask detecting unit 103, the reference point detecting unit 104, the distance calculating unit 105, the motion estimating unit 106, the awakening level decrease state estimating unit 107, the output unit 108, and the hand detecting unit 113 are included in the awakening effort motion estimation device 1c.

It is not limited to this, and some of the captured image acquiring unit 101, the face detecting unit 102, the mask detecting unit 103, the reference point detecting unit 104, the distance calculating unit 105, the motion estimating unit 106, the awakening level decrease state estimating unit 107, the output unit 108, and the hand detecting unit 113 may be mounted on an in-vehicle device of a vehicle, and the others may be included in a server connected to the in-vehicle device via a network. In this manner, the in-vehicle device and the server may constitute an awakening effort motion estimating system.

In addition, all of the captured image acquiring unit 101, the face detecting unit 102, the mask detecting unit 103, the reference point detecting unit 104, the distance calculating unit 105, the motion estimating unit 106, the awakening level decrease state estimating unit 107, the output unit 108, and the hand detecting unit 113 may be included in the server.

As described above, according to the fourth embodiment, the awakening effort motion estimation device 1c includes the hand detecting unit 113 that detects, on the basis of a captured image acquired by the captured image acquiring unit 101, whether or not an occupant's hand is present within a detection range of the occupant's face in the captured image, in which the motion estimating unit 106 does not estimate whether or not the occupant is performing an awakening effort motion by moving his or her mouth until the estimated stop time elapses in a case where the hand detecting unit 113 detects that the occupant's hand is present within the detection range of the occupant's face. Therefore, the awakening effort motion estimation device 1c can reduce erroneous estimation of the awakening effort motion by moving the driver's mouth, and improve estimation accuracy of the awakening effort motion.

In addition, the embodiments can be freely combined to each other, any constituent element in each of the embodiments can be modified, or any constituent element in each of the embodiments can be omitted.

### INDUSTRIAL APPLICABILITY

The awakening effort motion estimation device of the present disclosure can estimate that an occupant is performing an awakening effort motion by moving his or her mouth even when the occupant wears a mask.

### REFERENCE SIGNS LIST

1, 1a, 1b, 1c: awakening effort motion estimation device, 2: camera, 3: vehicle, 101: captured image acquiring unit, 102: face detecting unit, 103: mask detecting unit, 104: reference point detecting unit, 105: distance calculating unit, 106: motion estimating unit, 107: awakening level decrease state estimating unit, 108: output unit, 109: face direction detecting unit, 110: distance correcting unit, 111: mask size detecting unit, 112: adjustment unit, 113: hand detecting unit, 401: processing circuit, 402: input interface device, 403: output interface device, 404: processor, 405: memory

1. An awakening effort motion estimation device comprising:
processing circuitry configured to
acquire a captured image obtained by imaging an occupant's face in a vehicle;
detect, on a basis of the captured image having been acquired, two reference points for estimating a motion of the occupant's mouth, the two reference points including one point on a mask worn by the occupant, and the other point being a point based on a feature point of the occupant's face or a point different from the one point on the mask in the captured image;
calculate a reference point distance between the detected two reference points; and
estimate whether or not the occupant is performing an awakening effort motion by moving his or her mouth depending on whether or not the calculated reference point distance satisfies an awakening effort estimating condition.

2. The awakening effort motion estimation device according to claim 1, wherein
the processing circuitry estimates that the occupant is performing the awakening effort motion by moving his or her mouth in a case where a state where the reference point distance is equal to or more than a yawning determination threshold continues for equal to or more than a first determination time or in a case where a periodic change of the reference point distance continues for equal to or more than a second determination time and less than a third determination time longer than the second determination time.

3. The awakening effort motion estimation device according to claim 1, wherein
the two reference points are one of points based on feature points of the occupant's face and one point of a lower end of the mask.

4. The awakening effort motion estimation device according to claim 1, wherein
the two reference points are one point of an upper end of the mask and one point of a lower end of the mask.

5. The awakening effort motion estimation device according to claim 1, wherein
the two reference points are one point of an upper end of the mask and a feature point of the occupant's face indicating the driver's jaw.

6. The awakening effort motion estimation device according to claim 1,
wherein the processing circuitry is further configured to
detect a direction of the occupant's face on a basis of the captured image having been acquired;
correct the calculated reference point distance on a basis of the detected direction of the occupant's face; and
the estimate whether or not the occupant is performing the awakening effort motion by moving his or her mouth depending on whether or not the corrected reference point distance satisfies the awakening effort estimating condition.

7. The awakening effort motion estimation device according to claim 2,
wherein the processing circuitry is further configured to
detect a size of the mask on a basis of a distance between one point of an upper end of the mask and one point of a lower end of the mask; and
calculate a size of the occupant's face from a feature point of the occupant's face based on the captured image having been acquired and adjust the yawning determination threshold on a basis of the calculated size of the occupant's face and the detected size of the mask; and
estimate that the occupant is performing the awakening effort motion by moving his or her mouth in a case where a state where the reference point distance is equal to or more than the adjusted yawning determination threshold continues for equal to or more than the first determination time or in a case where a periodic change of the reference point distance continues for equal to or more than the second determination time and less than the third determination time longer than the second determination time.

8. The awakening effort motion estimation device according to claim 1,
wherein the processing circuitry is further configured to detect, on a basis of the captured image having been acquired, whether or not the occupant's hand is present within a detection range of the occupant's face in the captured image, and
the processing circuitry does not estimate whether or not the occupant is performing the awakening effort motion by moving his or her mouth until an estimated stop time elapses in a case where the processing circuitry detects that the occupant's hand is present within the detection range of the occupant's face.

9. An awakening effort motion estimation method comprising:
acquiring a captured image obtained by imaging an occupant's face in a vehicle;
detecting on a basis of the captured image having been acquired, two reference points for estimating a motion of the occupant's mouth, the two reference points including one point on a mask worn by the occupant, and the other point being a point based on a feature point of the occupant's face or a point different from the one point on the mask in the captured image;
calculating a reference point distance between the detected two reference points; and
estimating whether or not the occupant is performing an awakening effort motion by moving his or her mouth depending on whether or not the calculated reference point distance satisfies an awakening effort estimating condition.
